(19) United States
(12) Patent Application Publication
(10) Pub. No.: US 2023/0357064 A1
(43) Pub. Date: Nov. 9, 2023

(54) ENZYME BOOSTER TECHNOLOGY FOR ADVANCED DECONTAMINATION OF PETROLEUM HYDROCARBONS

(71) Applicants: Satinder Kaur Brar, Toronto (CA); Saba Miri, Toronto (CA); Seyyed Mohammadreza Davoodi, Toronto (CA)

(72) Inventors: Satinder Kaur Brar, Toronto (CA); Saba Miri, Toronto (CA); Seyyed Mohammadreza Davoodi, Toronto (CA)

(21) Appl. No.: 18/313,204

(22) Filed: May 5, 2023

Related U.S. Application Data
(60) Provisional application No. 63/339,357, filed on May 6, 2022.

(51) Int. Cl. C02F 3/34; C12P 21/00; C12M 1/00; C12N 9/02; B09C 1/10

(52) U.S. Cl. CPC C02F 3/342; C12P 21/00; C12M 21/14; C12M 23/52; C12N 9/0071; C12N 9/001; C12Y 103/01029; C12N 9/0069; C12Y 113/11001; C12Y 113/11002; C12N 9/0073; C12Y 114/13001; C12N 9/0008; C12Y 102/01065; C12N 9/0004; B09C 1/10; B09C 2101/00; C02F 2101/327

(57) ABSTRACT

Described herein is a PAH-degrading enzyme mixture obtained from a culture of PAH-utilizing microorganisms having been grown in presence of one or more enzyme inducers. Related methods and devices are also described.

### CROSS-REFERENCE TO RELATED APPLICATIONS

This application claims priority from a provisional application U.S. application No. 63/339,357 filed May 6, 2022, which is hereby incorporated by reference.

### FIELD

The present invention relates to petroleum hydrocarbons. In particular, the present invention relates to compositions and methods for the decontamination of petroleum hydrocarbons.

### BACKGROUND

Between 0.47 to 8.4 million tonnes of oil are released to seas or oceans each year, depending on the frequency and severity of oil spills. Spilled oil reaches lakes, rivers, wetlands, and freshwater environments and can harm all organisms that live in, under, or around the water (direct and indirect effects on human health).

Over the past decade, various technologies such as chemical methods, physical treatments, thermal treatments, and bioremediation have been applied to groundwater, soils, and sediments clean-up. Bioremediation technologies offer the potential for significant cost savings and practical approaches compared to conventional remediation technologies, such as thermal treatment, excavation, and disposal in landfills, and pump & treat. Especially at remote sites in the north, conventional technologies are often not practical or feasible, for example requiring electricity provision, transport of chemicals, and their storage, among others.

Petroleum degrading bacteria can produce specific enzymes that can degrade petroleum hydrocarbons. Commonly, the best (optimized) scheme of biodegradation cannot be easily achieved since the biodegradation efficiency of contaminants is strongly influenced by the physicochemical characteristics of the pollutant, the contaminated matrices, and microbial growth condition. Hence, the biochemical remediation method based on using enzymes, rather than whole-cell degrading microorganisms, can be a promising method for cold-climate sites since the enzymatic method can specifically catalyze a series of reactions for pollutant removal. The enzyme cocktail can be particularly formulated for a contaminated site based on physicochemical characteristics of hydrocarbon contaminated soil/water matrices. It would also be desirable to provide an enzyme cocktail and method for the degradation of crude oil, diesel, and polycyclic aromatic hydrocarbons.

In addition, the development of devices/processes to target oil below the water surface has attracted the attention of researchers due to the complication that started to emerge recently. For example, the explosion in tar sands productions in Western Canada means increasing amounts of heavy crude oil making its way to the American Midwest via the Great Lakes.¹ For these types of scenarios, the development of efficient remediation devices and/or processes is necessary to capture the plumes of the oil in the deep-water and limit the oil-impacted seafloor. In addition to extracting free phase hydrocarbon out of the water, the removal of aromatic hydrocarbons released into the water is important to avoid sediment contamination.

There remains a need for safe and efficient methods for managing contaminated sites.

### SUMMARY OF THE INVENTION

In accordance with an aspect, there is provided a polyaromatic hydrocarbon (PAH)-degrading enzyme mixture obtained from a culture of PAH-utilizing microorganisms having been grown in presence of one or more enzyme inducers.

In an aspect, the culture is a mixed-culture of two or more PAH-utilizing microorganisms.

In an aspect, the PAH-utilizing microorganisms are bacteria that are capable of growing with a PAH-containing composition being their only source of carbon.

In an aspect, the PAH-utilizing microorganisms are indigenous to a PAH-contaminated soil sample.

In an aspect, the PAH-utilizing microorganisms comprise Pseudomonas sp. and/or Rhodococcus sp.

In an aspect, the PAH-utilizing microorganisms comprise Pseudomonas sp. URS-5, Pseudomonas sp. URS-6, Pseudomonas sp. URS-8, and/or Rhodococcus sp. URS-10.

In an aspect, the one or more enzyme inducers comprise a mixture of inducers.

In an aspect, the mixture of inducers is comprised in oil.

In an aspect, the one or more enzyme inducers comprise pure inducers.

In an aspect, the one or more enzyme inducers comprise naphthalene, anthracene, phenanthrene, pyrene, benzo[a]pyrene, Dilbit, and/or crude oil.

In an aspect, the PAH-degrading enzyme mixture comprises an optimum active enzyme temperature of less than about 30° C., such as less than about 25° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., and any range therein between, such as from about 10° C. to about 15° C.

In an aspect, the PAH-degrading enzyme mixture comprises an optimum pH of from about 4 to about 9, such as from about 4, about 5, about 6, about 7, or about 8 to about 5, about 6, about 7, about 8, or about 9, such as about 4, about 5, about 6, about 7, about 8, or about 9, such as about 7.

In an aspect, the PAH-degrading enzyme mixture comprises less than about 10% salt or for use in an environment containing less than about 10% salt, such as less than about 9%, less than about 8%, less than about 7%, less than about 6%, less than about 5%, less than about 4%, less than about 3%, less than about 2%, or less than about 1% salt.

In an aspect, the enzymes are adsorbed on soil particles.

In an aspect, the PAH-degrading enzyme mixture further comprises a dispersing aid, such as a surfactant or biosurfactant, such as a rhamnolipid.

In an aspect, the enzymes comprise naphthalene dioxygenase, naphthalene cis-dihydridiol dehydrogenase, dihydrodiol dehydrogenase, catechol 1,2 dioxygenase, catechol 2,3 dioxygenase, salicylaldehyde dehydrogenase, 1-hydroxy-2-naphthoate hydroxylase, salicylate hydroxylase, trans-2-carboxybenzalpyruvate hydratase-aldolase, lipase, toluene monooxygenase, lignin peroxidase, manganese peroxidase, esterase, and/or laccase.

In an aspect, the PAH-degrading enzyme mixture further comprises one or more species of live PAH-utilizing organisms.

In accordance with an aspect, there is provided Pseudomonas sp. URS-5, Pseudomonas sp. URS-6, Pseudomonas sp. URS-8, and/or Rhodococcus sp. URS-10.

In accordance with an aspect, there is provided a method for tailoring a polyaromatic hydrocarbon (PAH)-degrading enzyme mixture for a selected contaminated site for bioremediation, the method comprising:

isolating indigenous PAH-utilizing microorganisms from the contaminated site,

culturing the microorganisms in the presence of one or more enzyme inducers,

extracting enzymes from the culture, and

applying the enzymes to the contaminated site.

In an aspect, the method comprises culturing a multi-culture of two or more PAH-utilizing microorganisms.

In an aspect, the PAH-utilizing microorganisms are bacteria that are capable of growing with a PAH-containing composition being their only source of carbon.

In an aspect, the contaminated site comprises soil. In an aspect, the PAH-utilizing microorganisms comprise Pseudomonas sp. and/or Rhodococcus sp.

In an aspect, the PAH-utilizing microorganisms comprise Pseudomonas sp. URS-5, Pseudomonas sp. URS-6, Pseudomonas sp. URS-8, and/or Rhodococcus sp. URS-10.

In an aspect, the one or more enzyme inducers comprise a mixture of inducers.

In an aspect, the mixture of inducers is comprised in oil. In an aspect, the one or more enzyme inducers comprise pure inducers.

In an aspect, the one or more enzyme inducers comprise naphthalene, anthracene, phenanthrene, pyrene, benzo[a]pyrene, Dilbit, and/or crude oil.

In an aspect, the PAH-degrading enzyme mixture comprises an optimum active enzyme temperature of less than about 30° C., such as less than about 25° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., and any range therein between, such as from about 10° C. to about 15° C.

In an aspect, the PAH-degrading enzyme mixture comprises an optimum pH of from about 4 to about 9, such as from about 4, about 5, about 6, about 7, or about 8 to about 5, about 6, about 7, about 8, or about 9, such as about 4, about 5, about 6, about 7, about 8, or about 9, such as about 7.

In an aspect, the contaminated site comprises less than about 10% salt, such as less than about 9%, less than about 8%, less than about 7%, less than about 6%, less than about 5%, less than about 4%, less than about 3%, less than about 2%, or less than about 1% salt.

In an aspect, the enzymes are adsorbed on soil particles.

In an aspect, the PAH-degrading enzyme mixture comprises a dispersing aid, such as a surfactant or biosurfactant, such as a rhamnolipid.

In an aspect, the enzymes comprise naphthalene dioxygenase, naphthalene cis-dihydridiol dehydrogenase, dihydrodiol dehydrogenase, catechol 1,2 dioxygenase, catechol 2,3 dioxygenase, salicylaldehyde dehydrogenase, salicylate hydroxylase, trans-2-carboxybenzalpyruvate hydratase-aldolase, lipase, toluene monooxygenase, lignin peroxidase, manganese peroxidase, esterase, and/or laccase.

In an aspect, the method further comprises applying one or more species of live PAH-utilizing organisms to the contaminated site.

In accordance with an aspect, there is provide a PAH-degrading enzyme mixture made by the method described herein.

In accordance with an aspect, there is provided a jellyfish-like device for bioremediation of a contaminated water site, the device comprising:
an enzyme reservoir containing PAH-degrading enzymes, and
a hollow fiber module in fluid communication with the enzyme reservoir,
wherein the hollow fiber module is permeable to PAHs but not to the PAH-degrading enzymes.

In an aspect, the PAH-degrading enzymes comprise the enzyme mixture described herein.

In an aspect, the hollow fiber module comprises hydrophilic modified polyethersulfone (mPES) hollow fibers.

In an aspect, the hollow fiber module comprises from about 1 to about 100 fibers, such as from about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, or about 95 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 fibers, such as about 10 fibers.

In an aspect, each fiber independently has an internal diameter of from about 0.1 mm to about 10 mm, such as from about 0.1, about 0.2, about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, or about 5.5, about 6.0, about 6.5, about 7.0, about 7.5, about 8.0, about 8.5, about 9.0, or about 9.5 to about 0.2, about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, or about 5.5, about 6.0, about 6.5, about 7.0, about 7.5, about 8.0, about 8.5, about 9.0, about 9.5, or about 10 mm, such as about 1.0 mm.

In an aspect, each fiber independently has a length of from about 10 to about 100 cm, such as from about 10, about 20, about 30, about 40, about 50, about 60, about 70, about 80, or about 90 to about 20, about 30, about 40, about 50, about 60, about 70, about 80, about 90, or about 100 cm, such as about 45 cm.

In an aspect, each fiber independently has a wall thickness of from about 0.01 mm to about 1 mm, such as from about 0.01, about 0.02, about 0.03, about 0.04, about 0.05, about 0.06, about 0.07, about 0.08, about 0.09, about 0.1, about 0.15, about 0.2, about 0.25, about 0.3, about 0.35, about 0.4, about 0.45, about 0.50, or about 0.55, about 0.6, about 0.65, about 0.7, about 0.75, about 0.8, about 0.85, about 0.9, or about 0.95 to about 0.02, about 0.03, about 0.04, about 0.05, about 0.06, about 0.07, about 0.08, about 0.09, about 0.1, about 0.15, about 0.2, about 0.25, about 0.3, about 0.35, about 0.4, about 0.45, about 0.50, or about 0.55, about 0.6, about 0.65, about 0.7, about 0.75, about 0.8, about 0.85, about 0.9, about 0.95, or about 1.0 mm, such as about 0.1 mm.

In an aspect, each fiber independently has a total surface area of from about 10 cm2 to about 100 cm², such as from about 10, about 20, about 30, about 40, about 50, about 60, about 70, about 80, or about 90 to about 20, about 30, about 40, about 50, about 60, about 70, about 80, about 90, or 100 cm2, such as about 58 cm².

In an aspect, the device further comprises a recycling pump, wherein the recycling pump optionally operates at a speed of from about 1 ml/min to about 100 ml/min, such as from about 1, about 2, about 3, about 4, about 5, about 6, about 7, about 8, about 9, about 10, about 20, about 30, about 40, about 50, about 60, about 70, about 80, or about 90 to about 2, about 3, about 4, about 5, about 6, about 7, about 8, about 9, about 10, about 20, about 30, about 40, about 50, about 60, about 70, about 80, about 90, or about 100, ml/min, such as about 50 ml/min.

In accordance with an aspect, there is provided a mobile production plant for producing the PAH-degrading enzymes described herein, the production plant comprising a fermenter for culturing microorganisms, a bioreactor for enzyme production, and an ultrasonic device for producing cell extracts.

In an aspect, the fermenter is a stirred tank fermenter.

In an aspect, the fermenter cultures the microorganisms at room temperature. In an aspect, the fermenter comprises pH, temperature, and/or dissolved oxygen (DO) probes.

In an aspect, the bioreactor operates in continuous mode.

In an aspect, the plant further comprises a power generator.

In an aspect, the plant further comprises an oxygen source such as an oxygen cylinder.

In an aspect, the mobile production plant is provided on a trailer.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIG. 1. Degradation rate after 37 and 47 days of incubation on a rotary shaker at 15° C.

FIG. 2. Phylogenetic tree of URS-5, URS-6, URS-8, and URS-10 and other related bacterial genera. Bootstrap values are shown from 1,000 replicates. GeneBank accession numbers for the 16S rRNA sequence are shown in the bracket.

FIG. 3. Enzyme activity of produced mixture in mono and coculture mode.

FIG. 4. The fermentation process based on mixed culturing of selected strains: (I) 1L shake flask for seed culture; (2) hybrid mechanically stirred hybrid airlift reactors fermenters and (III) Ultrasonicc processor.

FIG. 5. Flow chart of EnBooT applications.

FIG. 6. Particle-size analysis of soil.

FIG. 7. Zeta potential of soil samples as a function of pH.

FIG. 8. FTIR of mineral soil samples.

FIG. 9. Hydraulic conductivity measurement set up.

FIG. 10. Relative concentration on the breakthrough curve for trace element (Br) and total protein in the effluent sample during enzyme injection.

FIG. 11. Schematic diagram of a single-fiber reactor used for permeation studies (a) co-enzyme and enzyme solution (b) substrate.

FIG. 12. Release of PAHs from silicone O-ring into 1 ml MiliQ water when shaking at 300 rpm. The data were normalized to the final saturation concentrations.

FIG. 13. Hollow fiber beaker device for enzymatic reaction; (1) enzyme reservoir, (2) inlet peristaltic pump, (3) hollow fiber module, (4) outlet peristaltic pumps.

FIG. 14. The schematic diagram for measuring the diffusive air flow rates.

FIG. 15. Permeation data for anthracene through hollow fiber in a single-fiber reactor at 15° C.

FIG. 16. Effect of polyethyleneimine (PEI) concentration on the retainment ratio of nicotinamide adenine dinucleotide (NAD+) in presence of different millimolar concentrations of NaCl.

FIG. 17. Enzyme activity-time profiles which were circulated through the lumen.

FIG. 18. Lineweaver-Burk plots of enzyme mixture in hollow fibers and free solution.

FIG. 19. Normalized concentration of anthracene in compartment I at various λ². (F=100, σ=1,R=10).

FIG. 20. Normalized concentration of anthracene in compartment II at various λ². (F=100, σ=1,R=10).

FIG. 21. Decrease of total PAHs concentrations with time for the device with and without electrospun fibers.

### DETAILED DESCRIPTION

Indigenous microbial communities are constantly adjusting in response to environmental conditions. As described herein, using indigenous microorganisms can solve most of the challenges associated with environmental factors (temperature, pH, and salinity). The indigenous bacteria can produce enzymes to degrade high molecular weight hydrocarbons and result in less toxic metabolites that are more bioavailable for other bacterial strains in the community.

Using enzymes overcomes limitations of the slow rate of bioremediation by the whole-cell microorganisms. Enzymes can specifically catalyze a series of reactions in a minute timescale for pollutant removal.

Described herein are oxidoreductase enzymes from indigenous bacteria which are formulated for application for biodegradation of PAHs in soil and water. Also described is formulating the stabilized enzymes for a specified application such as for the breakdown of other petroleum hydrocarbons in specific conditions.

In aspects, enzyme cocktails were designed for effective biodegradation of at least one compound of polyaromatic hydrocarbons such as naphthalene, anthracene, phenanthrene and pyrene.

In aspects, a degradation method comprises bacterial enzymes for treating one of the above-mentioned materials in water and soil matrices.

Formulated bacterial enzymes from indigenous microorganisms in the environmental setting for degradation of poly-aromatic hydrocarbons may be of interest to remediation companies. In addition, the enzyme mixture can be formulated for a specific petroleum hydrocarbon in a contaminated site. This enzyme mixture can be combined with commercialized remediation products such as adsorbents to increase the remediation efficiency. This enzymatic technology can speed up the biodegradation of harmful chemicals by lowering the toxicity of target contaminants and increasing their bioavailability for other bacterial strains in the environment. By reducing the toxicity of contaminants in the system, intrinsic bioremediation can be reduced from years to weeks.

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, is intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.,” is derived from the Latin exempli gratia and is used herein to indicate a non-limiting example. Thus, the abbreviation “e.g.,” is synonymous with the term “for example.” The word “or” is intended to include “and” unless the context clearly indicates otherwise.

“Active” or “activity” for the purposes herein refers to a biological activity of the compositions described herein, wherein “biological” activity refers to a biological function (either inhibitory or stimulatory) caused by the compositions.

### Compositions

The Enzyme Booster Technology (EnBooT) described herein was developed as a novel and green bioremediation method based on the combination of specifically formulated enzymes and highly efficient biosurfactants which can be easily applied for in-situ biodegradation in cold-climate regions. This technology can be adapted for other sites with harsh or mild conditions and products can be specifically tailored based on need.

Thus, in aspects, described herein is a polyaromatic hydrocarbon (PAH)-degrading enzyme mixture obtained from a culture of PAH-utilizing microorganisms having been grown in presence of one or more enzymes inducers.

In some aspects, the culture is a monoculture and in other aspects, the culture is a mixed one.

### Methods

Also described herein are methods of use of the PAH-degrading enzymes and methods method for tailoring a PAH-degrading enzyme mixture for a selected contaminated site for bioremediation. The tailoring method comprises, for example, isolating indigenous PAH-utilizing microorganisms from the contaminated site, culturing the microorganisms in the presence of one or more enzyme inducers, extracting enzymes from the culture, and applying the enzymes to the contaminated site.

The method may comprise culturing a monoculture or a mixed-culture of two or more PAH-utilizing microorganisms. Typically, the PAH-utilizing microorganisms are bacteria that are capable of growing with a PAH-containing composition being their only source of carbon. The contaminated site typically comprises soil.

Any species of PAH-utilizing microorganisms are within the scope described herein. Typically, the PAH-utilizing microorganisms comprise Pseudomonas sp. and/or Rhodococcus sp., such as, for example, Pseudomonas sp. URS-5, Pseudomonas sp. URS-6, Pseudomonas sp. URS-8, and/or Rhodococcus sp. URS-10.

Typically, one or more enzyme inducers comprise a mixture of inducers and, typically, the mixture of inducers is comprised of oil. One or more enzyme inducers comprise pure inducers. Examples of enzyme induce include, for example, naphthalene, anthracene, phenanthrene, pyrene, benzo[a]pyrene, Dilbit, and/or crude oil.

The PAH-degrading enzyme mixture typically comprises an optimum active enzyme temperature of less than about 30° C., such as less than about 25° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., and any range therein between, such as from about 10° C. to about 15° C. and an optimum pH of from about 4 to about 9, such as from about 4, about 5, about 6, about 7, or about 8 to about 5, about 6, about 7, about 8, or about 9, such as about 4, about 5, about 6, about 7, about 8, or about 9, such as about 7.

Typically, the contaminated site comprises less than about 10% salt, such as less than about 9%, less than about 8%, less than about 7%, less than about 6%, less than about 5%, less than about 4%, less than about 3%, less than about 2%, or less than about 1% salt.

In other aspects, the enzymes are adsorbed on soil particles.

In aspects, the PAH-degrading enzyme mixture comprises a dispersing aid, such as a surfactant or biosurfactant, such as a rhamnolipid.

Exemplary enzymes include naphthalene dioxygenase, pyrene dioxygenase, dihydrodiol dehydrogenase, 1-hydroxy-2-naphthoate hydroxylase, catechol 1,2 dioxygenase, catechol 2,3 dioxygenase, salicylate hydroxylase, lipase, toluene monooxygenase, lignin peroxidase, manganese peroxidase, esterase, and/or laccase.

In aspects, the method further comprises applying one or more species of live PAH-utilizing organisms to the contaminated site.

Also contemplated herein is a PAH-degrading enzyme mixture made by the method described herein.

The following examples do not include detailed descriptions of conventional methods. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

### Jellyfish-Like Device

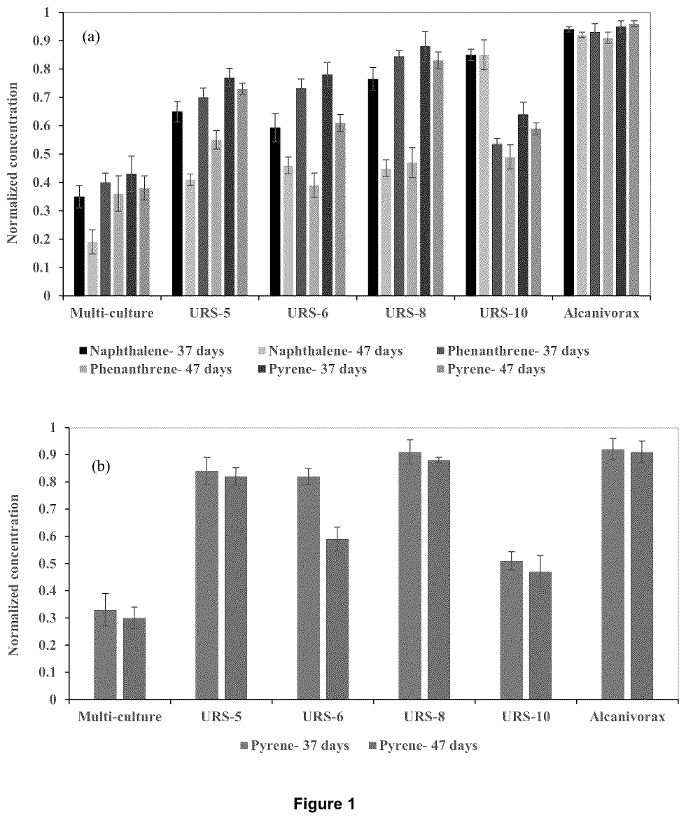

Further described herein is a jellyfish-like device for bioremediation of a contaminated water site. The device comprises an enzyme reservoir containing PAH-degrading enzymes, and a hollow fiber module in fluid communication with the enzyme reservoir, wherein the hollow fiber module is permeable to PAHs but not to the PAH-degrading enzymes. In typical aspects, the PAH-degrading enzymes comprise the enzyme mixture described herein (FIG. 1).

In aspects, the hollow fiber module comprises hydrophilic modified polyether sulfone (mPES) hollow fibers. The hollow fiber module typically comprises from about 1 to about 100 fibers, such as from about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, or about 95 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 fibers, such as about 10 fibers.

Typically, each fiber independently has an internal diameter of from about 0.1 mm to about 10 mm, such as from about 0.1, about 0.2, about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, or about 5.5, about 6.0, about 6.5, about 7.0, about 7.5, about 8.0, about 8.5, about 9.0, or about 9.5 to about 0.2, about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, or about 5.5, about 6.0, about 6.5, about 7.0, about 7.5, about 8.0, about 8.5, about 9.0, about 9.5, or about 10 mm, such as about 1.0 mm.

Typically, each fiber independently has a length of from about 10 to about 100 cm, such as from about 10, about 20, about 30, about 40, about 50, about 60, about 70, about 80, or about 90 to about 20, about 30, about 40, about 50, about 60, about 70, about 80, about 90, or about 100 cm, such as about 45 cm.

Typically, each fiber independently has a wall thickness of from about 0.01 mm to about 1 mm, such as from about 0.01, about 0.02, about 0.03, about 0.04, about 0.05, about 0.06, about 0.07, about 0.08, about 0.09, about 0.1, about 0.15, about 0.2, about 0.25, about 0.3, about 0.35, about 0.4, about 0.45, about 0.50, or about 0.55, about 0.6, about 0.65, about 0.7, about 0.75, about 0.8, about 0.85, about 0.9, or about 0.95 to about 0.02, about 0.03, about 0.04, about 0.05, about 0.06, about 0.07, about 0.08, about 0.09, about 0.1, about 0.15, about 0.2, about 0.25, about 0.3, about 0.35, about 0.4, about 0.45, about 0.50, or about 0.55, about 0.6, about 0.65, about 0.7, about 0.75, about 0.8, about 0.85, about 0.9, about 0.95, or about 1.0 mm, such as about 0.1 mm.

Typically, each fiber independently has a total surface area of from about 10 cm2 to about 100 cm², such as from about 10, about 20, about 30, about 40, about 50, about 60, about 70, about 80, or about 90 to about 20, about 30, about 40, about 50, about 60, about 70, about 80, about 90, or 100 cm², such as about 58 cm².

Typically, the device further comprises a recycling pump, wherein the recycling pump optionally operates at a speed of from about 1 ml/min to about 100 ml/min, such as from about 1, about 2, about 3, about 4, about 5, about 6, about 7, about 8, about 9, about 10, about 20, about 30, about 40, about 50, about 60, about 70, about 80, or about 90 to about 2, about 3, about 4, about 5, about 6, about 7, about 8, about 9, about 10, about 20, about 30, about 40, about 50, about 60, about 70, about 80, about 90, or about 100, ml/min, such as about 50 ml/min.

### Mobile Production Plant

To improve production efficiency resulting in cost-effective enzymes, an unsterile and continuous fermentation process was developed by using a compact production plant for increased responsiveness and mobility.

The enzymatic remediation of petroleum-contaminated sites is a promising approach for site clean-up. However, the cost and efficiency of the enzyme cocktails that are employed in these processes are still a significant bottleneck. In order to address issues regarding the clean-up of contaminated sites in cold regions, described herein are a mobile product plant and method for on-site production of enzyme solutions and biochemical products using a compact and mobile production plant in order to reduce the production cost of this technology. Typically, the mobile production plant comprises: (1) a 3 L customized stirred tank fermenter for culturing of strains at room temperature equipped with pH, temperature and DO probes; (2) a 15 L customize hybrid bioreactors applied for bench-scale enzyme production operating in continuous mode equipped 5-inch exhaust filter; and (3) an ultrasonic device used for pilot-scale production of cell extract. Piping would connect components. All these equipment as well as other additional equipment such as a power generator, oxygen cylinder, may be provided pre-packaged on a trailer, for increased mobility and responsiveness.

Based on the literature review², the most representative enzymes involved in bioremediation belong to the family of oxidoreductases. Kadri et. al (2018) studied bench-scale production of the cocktail enzymes using 5 L stirred tank reactor (STR) experiments. They showed that a significant decrease in the dissolved oxygen (DO) values was detected during the first stage of fermentation. The decrease in DO observed is coincidental with the exponential growth phase of strains that required a higher oxygen uptake rate (OUR). Considering the fact that maximum biomass and enzyme activities can be achieved in presence of high dissolved oxygen, a higher agitation from 250 to 500 rpm (representing the large power domain in the process) and a higher aeration rate reaching 3.5 L min-¹ were applied to maintain dissolved oxygen greater than 60%.³ The challenges to achieving effective and economical production become greater for larger volumes where utility costs generally dominate fermentation economics. The airlift bioreactor has the potential for use in the large-scale production of these enzymes due to its advantages over STR with respect to higher gas hold-up, higher mass transfer, lower shear stress, lower microbial contamination, lower energy consumption, installation, and operation costs. In addition, an airlift bioreactor can supply more oxygen, and efficiency of petroleum degrading enzymes is increased compared to stirred conventional reactors.⁴ Air-lift bioreactors have been widely used in a number of industrial processes, such as the production of amino acids, antibiotics, enzymes, vitamins, and organic acids. However, the continuous enzyme production in air-lift reactors was reported for several enzymes, to the best of our knowledge, there is no report available on the production of petroleum-hydrocarbon enzymes.

Petroleum hydrocarbons have a significant role in the production of degrading enzymes and using petroleum hydrocarbons as the sole source of carbon inhibits the growth of other non-degrading microorganisms. Due to the inhibitory and toxicity effect of such compounds, selective growth will be obtained with a specific C: N ratio of media for seed cultivation and enzyme production. Thus, a complicated facility operating in sterile conditions is not needed for the production of targeted enzymes because the specific conditions for the growth of specific bacteria are provided.

Thus, more generally, described herein is a mobile production plant. In aspects, the mobile production plant is for producing the PAH-degrading enzymes described herein. The production plant comprises a fermenter for culturing microorganisms, a bioreactor for enzyme production, and an ultrasonic device for producing cell extracts.

In certain aspects, the fermenter is a stirred tank fermenter and optionally cultures the microorganisms at room temperature. The fermenter typically comprises pH, temperature, and/or dissolved oxygen (DO) probes. Typically, the bioreactor operates in continuous mode.

In certain aspects, the mobile production plant further comprises a power generator and/or an oxygen source such as an oxygen cylinder. Typically, the mobile production plant is provided on a trailer.

Without further description, it is believed that one of ordinary skill in the art can, using the preceding description and the following illustrative examples, make and utilize the compositions of the present invention and practice the claimed methods. The following working examples, therefore, specifically point out the typical aspects of the present invention and are not to be construed as limiting in any way in the remainder of the disclosure.

### EXAMPLES

This study aims to investigate (1) the bench-scale production of aliphatic and PAHs degrading enzymes; (2) the feasibility study for soil application through bench-scale and soil column tests; and (3) the fabrication of jellyfish-like devices for marine spill clean-up. This device is inspired by the long, elegant appendages of jellyfish that use their sticky tentacles to catch the oils below the surface water during a passive process when they float into the piece of oil by an incident. Each device is composed of a recirculation reservoir (e.g., jellyfish bell), and hollow fiber membranes used for physical immobilization of PAH degrading enzymes.

Future work includes the application of formulated enzymes for the field scale tests (in-situ and ex-situ) and scale-up of enzyme production and combination of enzyme mixture with commercialized remediation products. In addition, the application of metagenomics will be used for the analysis of microbial community to understand the shift in microbial population because of detoxification of the contaminant in soil and degradation of polyaromatic hydrocarbons. This date will be used for the optimization of enzyme concentrations.

### Example 1: Isolation and Screening of Degrading Bacteria

### Laboratory Experiments for Enzyme production

Before inoculation for enzyme production, each bacterium was cultured in a nutrition broth (NB) at 15±1° C. and 150 rpm. A correlation was obtained between cell concentration and the number of viable bacteria by measuring optical density at 600 nm and counting colony-forming units (CFU).

The enzyme production based on monoculture of newly isolated was carried out in 250 mL serum bottles containing 50 mL of NB supplemented with 100, 250, and 500 ppm of oil. The target enzymes were also produced based on the mixed-culture of newly isolated Pseudomonas URS-5, URS-6, URS-8, Rhodococcus URS-10. In this regard, the enzyme production was carried out in 250 mL serum bottles containing 50 mL of NB supplemented with 250 mg/L of oil as a mixture of enzyme inducers. After 24h of incubation, the culture was centrifuged at 3810×g for 20 min. Cell extracts containing PAH degrading enzymes were prepared from 100 ml of each cultured Pseudomonas sp. URS-5, Pseudomonas sp. URS-6, Pseudomonas sp. URS-8, and Rhodococcus sp. URS-10 grown on oil. When growth reached the late exponential phase, cells were harvested from the media after culturing by centrifugation (16,000 rpm for 4 min at 4° C.). The pellets with the biomass were resuspended in phosphate buffer, pH 8, and then sonicated on ice using an Ultrasonifier (Branson Ultrasonics Corporation, Danbury, CT, USA) at 22 kHz and 30 kHz frequencies of ultrasounds for 10 min. Bradford assay was performed using Pierce TM BCA Protein Assay Kit (Thermo Fisher Scientific, Canada) and bovine serum albumin (BSA) standard curve.

### Assay for PAH Degrading Enzymes

Naphthalene dioxygenase activity is assayed based on the formation of indigo at 500 nm per time unit. The enzyme reaction was carried out using 5 μl of indole 100 mM as a substrate in DMF to the cellular lysates. The absorbance of the reaction mixture at 500 nm was recorded against a blank containing all ingredients except the substrate. The specific enzyme activity was defined as the initial rate of indigo formation by plotting the increase in absorbance at the first 2 h of reaction normalized to the protein content of the sample. The maximal absorbance of indigo was determined by mass spectrum at different wavelengths using a standard indigo solution dissolved in N,N′-Dimethyl-Formamide (DMF)⁵.

Naphthalene cis-dihydrodiol dehydrogenase activity was assayed by spectrophotometric measurement of the reduction of NAD at 340 nm. Routine assays were performed at 30° C. in a 1-ml quartz cuvette maintained under argon. The assay mixture (0.6 ml) contained 1 mM NAD and 0.1 mM dihydrodiol substrate in 0.1 M potassium phosphate, pH 7.0. The reaction was initiated by an appropriate amount of enzyme, and the absorption at 340 nm was recorded at 0.1-s intervals over 1 min with an HP8452 spectrophotometer equipped with a thermostated cuvette holder (Agilent Technologies, Les Ulis, France). The enzyme activity was calculated from the initial linear portion of the time course using an absorption coefficient of 6,220 M1·cm1 for nicotinamide adenine dinucleotide (NADH). One enzyme unit was defined as the amount that catalyzed the formation of one micromole of NADH per minute⁶.

Catechol 2, 3-dioxygenase and catechol 1, 2-dioxygenase activities are assayed by monitoring the production of 2-hydroxymuconic semialdehyde and muconic acid at 375 nm and 260 nm, respectively. The reaction mixture contained 1 mM of catechol, 2.0 mL of phosphate buffer (pH=7.0), and cellular lysates. One unit of enzyme activity is defined as the amount of enzyme that produced 1 μmol of cis, cis-muconic acid, and 2-hydroxy muconic semialdehyde per time unit.⁷

1-hydroxy-2-naphthoate and salicylate hydroxylase activities were measured at 340 nm by an assay described by Balashova et al.⁸ The reaction mixture (1 ml) contained 20 mM KH2PO4 (pH7.5). 100 μl cell extract, 100 μM NADH, and 50 μM 1-hydroxy-2-naphthoate or salicylate, respectively. Molar reaction coefficient at 340 nm of 5.08 and 6.22 mM−1cm⁻¹ were used to determine the reaction of 1-hydroxy-2 naphthoate and salicylate. The rate of change in light absorption was measured at 340 nm. One unit of activity is the amount of enzyme that is needed to catalyze the conversion of one mol of NADH per minute into oxygen.⁹

### Enzyme Cocktail Characterization

Pre-selected isolates were tested for the key enzymes involved in the degradation of target substrates. The involved enzymes identification is studied by carrying out LC-MS/MS to confirm the presence of target enzyme in enzyme mixture obtained from mixed-culture. To provide a reference data set for peptide spectrum match (PSM) analysis for target enzymes, an enzymes cocktail was prepared using the suspension trapping (S-Trap) method as described in detail in our previous study (Miri et al., 2021a). Resulted peptides were subjected to mass determination analysis using a mass spectrometer coupled to an EASY-nLC 1000 system (Thermo Fisher Scientific, USA) and mass spectrometry (MS)-MS sequence analysis using Orbitrap Elite mass spectrometer (Thermo Fisher Scientific, USA). All raw data were processed using the software Proteome Discoverer (version 2.2, Thermo Fisher Scientific, USA), then aligned with the protein database of Universal Protein Resource, UniProt (www.uniprot.org) and BLASTP (https://blast.ncbi.nlm.nih.gov).^{10, 11}.

Example of Process for Isolation of Bacteria for Producing Enzymes

A series of pyrene biodegradation tests were conducted using SSM supplemented with three PAHs (naphthalene, phenanthrene, pyrene); and among all the isolated obtained (28 strains), URS-5, URS-6, URS-8, and URS-10 were selected based on their ability to grow on pyrene as a sole carbon and energy source. The constant freely dissolved concentrations of PAHs were provided using O-ring silicone as described in the experimentation section. Positive biodegradation ability was confirmed by measuring the percent depletion of PAHs in the silicone O-ring after 37 and 47 days for the biodegradation experiments (FIG. 1).

A comparison of the degradation of pyrene by strains under a single and mixture substrate system was conducted in order to determine if the presence of other substrate would affect (either suppressing or enhancing) the degradation of the primary substrate. As can be seen in FIG. 1, URS-5, URS-6, URS-8, and URS-10 could degrade 27, 39, 17 and 41% of pyrene after 47 days. Alcanivorax sp. almost failed to degrade pyrene. Measurement of the PAH degradation rates showed that Pseudomonas URS-5, URS-6 and URS-8 showed less than 25 percent removal of pyrene in a mixture substrate system within 37 days at 10° C. The initial concentration was 18.31, 2.98, 1.01, 0.08 ppm of naphthalene, anthracene, phenanthrene, pyrene in the medium.

Pseudomonas URS-5, URS-6 could grow in the range of temperature between 4±1° C. and 25±1° C., but not as effectively at 37±1° C. as previously reported for P. synxantha and P. mandelii strains.⁷ The presence of these characteristics indicates that the strains do not pose any potential pathogenic risk to humans or animals. The partial 16S rRNA sequencing showed that URS-6 was close to the type of strain of Pseudomonas putida strain NR 043434.1T with 99% identity. The sequence identity of URS-5 and URS-8 were 99% with a type of strain of Pseudomonas mandelii strain Cl P 105273T and Pseudomonas syringae ATCC 19310T respectively. In the sequence analysis of URS-5, the sequence identity was 98% with a type of strain of Rhodococcus erythropolis JCM 3201T.

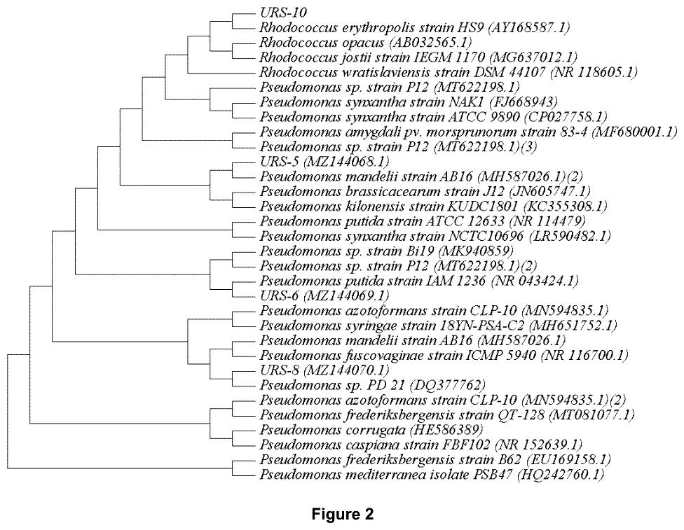

The sequences for URS-5, URS-6, URS-8, and URS-10 have been deposited in the NCBI Genbank database and were assigned the accession of MZ144068.1, MZ144069.1, MZ144070.1 and MZ959374, respectively. Phylogenetic analysis also grouped URS-5, URS-6, URS-8 and URS-10 with the strain of Pseudomonas putida, Pseudomonas mandelii (P. mandelii), Pseudomonas synxantha, an Rhodococcus erythropolis with 98% bootstrap support (FIG. 2).

Pseudomonas spp. and Rhodococcus spp. remarkably degrade various xenobiotics and often are isolated from hydrocarbon-contaminated sites.¹² Wald et. al 2015 indicated that the Pseudomonas syringae group (namely Pseudomonas cichorii LMG 2162T) was one of the most dominant Pseudomonads in the sequestration and of carbon from naphthalene in the samples and potential degradation of other PAHs upon aeration of the sediment at both 20 (7%) and 10° C.¹³ Margesin et al., (2008) isolated cold-tolerant strains from contaminated soil which can degrade BTEX at 10° C.¹⁴

### Determination of Pyrene Degradation Rate by Individual Strain and Multi-Culture Strains

Regarding the URS-5, general sequental pattern of degradation was observed in which naphthalne was transformed first, followed by phenanthrene, and pyrene (FIG. 1(a)). This was previously observed for other dioxygenases from Pseudomonas sp. strain NCB 9816-4 and Sphingomonas CHY-1strain that the enzymes in the upper part of the naphthalene pathway possess broad substrate specificities.^{15, 16} This pattern of degradation also agrees with those of previous studies in which degradation rates generally decreased with increasing molecular weight. This has been attributed to the aqueous solubility of compounds. Specifically, lower molecular weight PAHs are more soluble and bioavailable; therefore, they are used first by the microorganisms. Moreover, the present study showed simple degradation pathway and less metabolite produced compared to other isolated. The lack of accumulated intermediate for the degradation of PAHs in samples can be attributed to the fact that the rate-limiting step for the biodegradation might be the initial ring oxidation reaction. The other reason of simple degradation pathway might be the enzyme activities observed for the oxidation of NADH in the presence of salicylate and 1-hydroxy-2-naphthoate hydroxylase in the cultures grown on three PAHs. As a consequence, these findings suggest that phenanthrene and pyrene are degraded by the enzymes involved in the naphthalene degradative pathway.

Regarding the URS-8, the same pattern of degradation for pyrene was observed. This strain did not show extensive degradation of pyrene in the PAHs mixture; however, the lack of accumulated intermediate can be considered positive effect that might be attributed to the enzyme system presented in liquid medium. The lack of salicylate hydroxylase activity in cell free lysate suggesting a pathway for three PAHs that does not pass through salicylate. However, protocatechuic acid 3,4-dioxygenase enzyme assay suggesting the pathway for the catabolism of phthalate to central metabolites via intradiol cleavage of protochatechuic acid (Table.1). Other studies reported the relationship between degradation rate of PAHs and naphthalene dioxygenase activity.¹⁷

Compared to URS-5 and URS-8 strains, URS-6 showed higher degradation rate that might be attributed to increasing the bioavailability of pyrene as well as high activity of naphthalene dioxygenase enzyme (FIG. 1). This isolate was considered as efficient biosurfactant producers based on surface tension measurement. The solubility of PAHs including phenanthrene and pyrene could potentially be improved by a biosurfactant, which would in turn increase the rate of biodegradation.

For URS-10, differet pattern of degradation was observe in which this strain degraded small amount of pyrene in the PAHs mixture, and degraded considerably more pyrene when it was present as the sole subtrate (FIG. 1). These results suggested the presence of pyrene degrading enzymes repressed in culture grown in the mixture of PAHs. The enzyme activity of pyrene dioxygenase was calculated using the partially purified dioxygenase and measuring substrate-dependent oxidation of NADH (Table 1). Naphthalene did not appear to be transformed by this dioxygenase and no particular reaction was observed when indole was added to the reaction. There is a possibility that a negative result for a given PAH can be attributed to undetermined factors other than substrate specificity (e.g., poor expression, enzyme instability).¹⁸

As shown in FIG. 1, the degradation effect of mixed bacteria compoased of pre-selected isolated bacteria and Alcanivorax borkumensis was better than pure cultures. Adding biosurfactant-producing bacteria i.e., Alcanivorax borkumensis and URS-6 increased the bioavailability of the pyrene and phenanthrene. Moreover, in more complicated system the presence of other PAHs had no effect on the degradation of pyrene that might be attributed to the comprehensive PAH-degradation capability, Intermediate study showed a buildup of xenobiotic compounds concentration i.e., phthalic acid while monitoring the degradation of pyrene by URS-10 that could be attributed to the lack of phthalic acid mineralizing enzymes. This implied the importance of consortium where phthalic acid is not a dead-end product of bacterial consortia metabolism.

### Production of Involved Enzymes in Erlenmeyer flasks

The above-mentioned bacterial strains, including but not limited to, Pseudomonas, and Rhodococcus, can be mixed culturing for enzyme production. Two types of fermentation media (minimum salt media (MSM) and nutrition broth (NB)) are compared for enzyme production. The NB media contains all the elements for growth and is not selective and the MSM media can provide inorganic nutrients for the growth of bacteria. Both media are supplemented with 50-500 mg/L of crude oil as a mixture of enzyme inducers. Using standard media can support the high-cell density of cultures and can provide all needed nutrition of mixed-culture of bacterial strains. For example, produced biomass in NB is around 5-fold higher than MSM, and enzymes are produced 10-fold higher in NB compared to MSM. Using pure inducers (such as naphthalene, anthracene, phenanthrene, pyrene, and benzo[a]pyrene) results in the induction of certain enzymes NOT all the target enzymes. For example, naphthalene can induce the production of naphthalene dioxygenase and catechol 2,3 dioxygenase in Pseudomonas strains NOT lipase and toluene monooxygenase.

Crude oil assimilating resting cells including Pseudomonas sp. URS-5, Pseudomonas sp. URS-6, Pseudomonas sp. URS-8, and Rhodococcus sp. URS-10 was applied for the production of PAH degrading enzymes. The time-course experiments showed that 37 hours of incubation might be the best time when the production of enzymes is close to maximum when growth reaches its stationary phase. Ultrasonic dispersion is a bioavailability improvement method used prior to incubation to reach favorable biodegradation efficiency by PAH-utilizing microorganisms. Table 1 showed the results of cell lysates from cultured bacteria in NB supplemented with 100, 250 and 500 mg l⁻¹ of crude oil as inducer. As seen in this table, the concentration of the supplement (inducers) is one of the determining factors in enzyme expression⁷. The cell lysate from cultured Pseudomonas sp. URS-6 in the presence of 250 mg l⁻¹ of crude oil showed high naphthalene dioxygenase and catechol 2,3 dioxygenase activity, while cell extract from Pseudomonas sp. URS-8 and URS-5 showed high Protocatechuic acid 3,4-dioxygenase, and 1-hydroxy-2-naphthoate hydroxylase activity, respectively. Rhodococcus sp. URS-10 showed high activity of pyrene dioxygenase. Moreover, the extract cells from the cultured bacteria in the presence of 100, 250 and 500 mg l⁻¹ of crude oil showed a significant difference in activity of the enzyme in order of 500>250>100 (p<0.05), and 250 mg l⁻¹ was the optimal concentration for enzyme production.

TABLE 1
Enzyme determination in Pseudomonas sp. URS-5, Pseudomonas sp. URS-6,
Pseudomonas sp. URS-8, and Rhodococcus sp. URS-10. (T = 10° C., pH = 7, Salinity = 10%)

| | | Enzyme activity with | | |
|---|---|---|---|---|
| | | induction of crude oil | | |
| Name of strain | Produced enzymes | 100 ppm | 250 ppm | 500 ppm |
| Pseudomonas sp. URS-5 | Naphthalene dioxygenase | 3.2ª ± 0.3 | 4.7 ± 0.5 | 1.8 ± 0.1 |
| | Catechol 2,3 dioxygenase | 13.3 ± 0.1 | 14.7 ± 0.5 | 14.4 ± 0.2 |
| | 1-hydroxy-2-naphthoate hydroxylase | 5.9 ± 0.3 | 5.5 ± 0.3 | 2.9 ± 0.9 |
| | Salicylate hydroxylase | 4.5 ± 0.1 | 4.8 ± 0.4 | 3.9 ± 0.1 |
| Pseudomonas sp. URS-6 | Naphthalene dioxygenase | 9.8 ± 1.3 | 12.3 ± 0.9 | 11.9 ± 0.6 |
| | Catechol 1,2 dioxygenase | 13.4 ± 0.4 | 17.6 ± 0.5 | 9.1 ± 0.1 |
| | Catechol 2,3 dioxygenase | 1.9 ± 0.3 | 2.5 ± 0.4 | 1.2 ± 0.7 |
| | 1-hydroxy-2-naphthoate hydroxylase | 3.1 ± 0.7 | 4.8 ± 0.4 | 3.7 ± 0.5 |
| | Salicylate hydroxylase | 5.3 ± 0.4 | 5.6 ± 0.2 | 3.9 ± 0.7 |
| Pseudomonas sp. URS-8 | Naphthalene dioxygenase | 3.6 ± 0.3 | 4.1 ± 0.6 | 3.3 ± 0.1 |
| | Catechol 2,3 dioxygenase | ND | 0.13 ± 0.05 | ND |
| | Protocatechuic acid 3,4-dioxygenase | 4.3 ± 0.4 | 5.4 ± 1.4 | 3.8 ± 0.5 |
| Rhodococcus sp. URS-10 | Pyrene dioxygenase | 14.5 ± 0.1 | 16.3 ± 0.5 | 12.5 ± 0.3 |

As discussed previously, removal of more than 50% is achieved for targeted PAHs in the multi-culture of bacteria than the monoculture of isolated strain (FIG. 2). Thus, the enzyme mixture was obtained via multi-culture of isolated strains, and it was characterized by tandem LC-MS/MS.

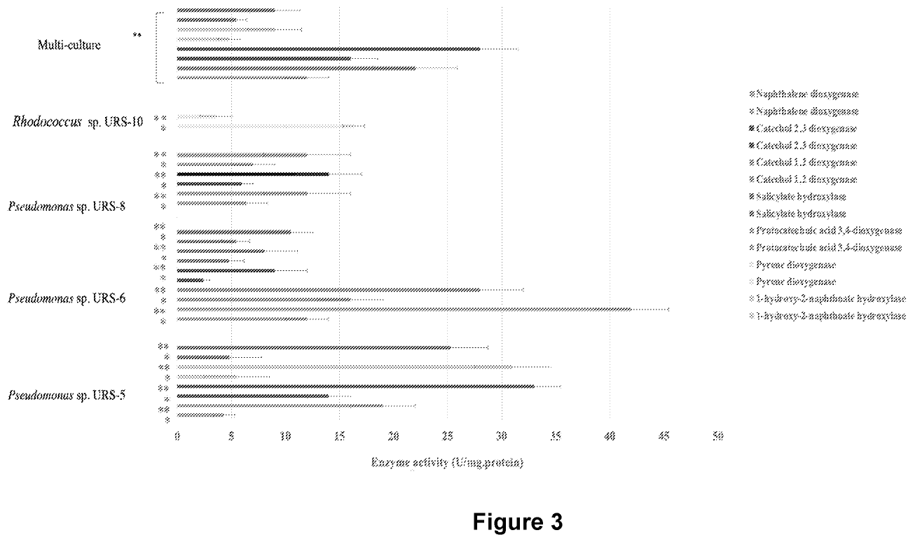

The production of target enzymes was carried out in 250 ml serum bottles containing 50 ml of NB supplemented with 250 mg l⁻¹ of crude oil. As can be seen in FIG. 3, the results showed that the enzyme production increased in the multi-culture of selected strains. Some literature proposed using genetically engineered microorganisms to obtain preferable biodegradation pathways in a single strain; however, these enzymes can be brought together from different bacteria to design multi-step catalysis of PAHs degradation. Moreover, environmental risks of the potential release of genetically engineered microorganisms in the environments need to be considered².

### Production of Involved Enzymes in the Fermenter

Large-scale production of EnBooT revealed that the yield coefficients (overall specific cell yield on substrate) and total productivity cannot reach their highest theoratical values with the cultivation at current stirred tank fermenters in spite of controlling oxygen transfer through pressurization of the reactor, agitation, air inlet and feed rate. Stired-tank reactor is the most ccommonly used gas-liquid contactor that often cinsists of flat-blade turbine for gas dispersion and agitation. Traditionally, rushton turbine (RT) impeller used for the process of gas-liquid stirred reactor resulted in poor gas dispersibility in the regions far away from impeller due to limited gas handling capacity of RT impeller and drastic pressure. Thus, a custom- design bioreactor was needed to balance the culture's oxygen demand over the full biomass range thereby cells consume oxygen at such rate high rate that the maximum possible oxygen utilization value was approached. The multi-impeller stirred tank with draft tube design configuration consisting of an improved Rushton turbine combined with a pitched-blade turbine offers the advantage of enhanced media circulation, mixing and gas-liquid mass transfer coefficient for suspending solids (biomass). This fermentation has design criteria similar to mechanically stirred hybrid airlift reactors fermenters. The upper impeller is a down-pumping axial flow impeller (e.g., six-pitched blade disc turbine impeller). In order to achieve relatively high local gas holdup and long bubble residence time, the circulation of the fluid below the upper paddle is downward-directed, opposite to the mainstream of the bubble. The gas introduced from the gas sparger can be effectively dispersed along the radial direction above the bottom impeller (e.g. Punched rigid-flexible impeller) and rises upward in the annular region between the tank wall and the draft tube. Punched rigid-flexible impeller was proposed to improve the gas-liquid dispersion performance in the reactor.

### Compact Production Plant for On-Site Production of EnBooT

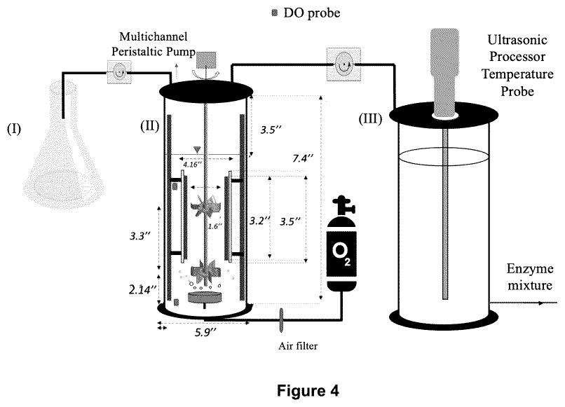

To improve production efficiency resulting in cheaper enzymes, a fermentation process was developed by using a compact production plant for maximum responsiveness and mobility (FIG. 4). As can be seen in this FIG. 4, the seed cultures were grown at 17° C. in NB medium for 12 h at 200 rpm on a rotary shaker. Subsequently, seed cultures in 5% volume were inoculated into the NB medium containing 250 ppm of crude oil.

### Determination of Involved Enzymes in the Mixture

To confirm the presence of target enzymes in the crude cocktail obtained from mixed-culture of PAH-degrading bacteria, the liquid chromatography-Tandem mass spectrometry (LC-MS/MS) was carried out. This method can provide substantially more information in the identification of enzymes in a mixture compared to conventional biochemical assays¹⁰. The identified proteins with more than 1% false discovery rate (FDR) are listed in Table 2. Five PAH-degrading enzymes were identified: naphthalene dioxygenase, naphthalene 1,2-dioxygenase, dihydrodiol dehydrogenase, catechol-2,3-dioxygenase, salicylaldehyde dehydrogenase. The sequence of the crude cocktail showed the presence of other proteins (such as membrane proteins, stress-response proteins, ATP-binding proteins, etc.), however, they did not show strict identity to the sequences of any proteins of known PAH-degrading function in the mixture.

TABLE 2
Identification of partially purified enzymes by LC-MS/MS analysis
and assignment to the corresponding UniProtKB entry.

| | | | Mass¹ | Mass² | | Coverage |
|---|---|---|---|---|---|---|
| Protein/Enzyme | Molecular function | Gene | (kDa) | (kDa) | UniProt ID | (%) |
| Naphthalene | Component of the | doxB | 49.6 | 49.1 | P0A111 | 85 |
| dioxygenase | naphthalene | | | | | |
| | dioxygenase | | | | | |
| Naphthalene 1,2- | Ferredoxin | doxA | 11.4 | 11.8 | P0A170 | 81 |
| dioxygenase | component of the | | | | | |
| | naphthalene | | | | | |
| | dioxygenase | | | | | |
| Dihydrodiol | The oxidation of | nahB | 27.5 | 27.4 | P0A169 | 79 |
| dehydrogenase | naphthalene | | | | | |
| | dihydrodiol | | | | | |
| Catechol-2,3- | The meta | catE | 31.5 | 31.2 | P54721 | 61 |
| dioxygenase | cleavage of | | | | | |
| | catechol to 2- | | | | | |
| | hydroxymuconic | | | | | |
| | semialdehyde | | | | | |
| ATP synthase subunit | Produces ATP | atpD | 51.9 | 51 | C1A1X8 | 57 |
| beta | from ADP | | | | | |
| Salicylaldehyde | Involves in | doxF | 51.9 | 52 | P0A390 | 58 |
| dehydrogenase | pathway | | | | | |
| | naphthalene | | | | | |
| | degradation | | | | | |
| Alkyl hydroperoxide | Protects the cell | ahpF | 55.3 | 55.8 | P0A155 | 56 |
| reductase | against DNA | | | | | |
| | damage | | | | | |
| Chaperone protein | Part of a stress- | clpB | 92.3 | 93 | I3UXE4 | 51 |
| ClpB | induced multi- | | | | | |
| | chaperone | | | | | |
| | system | | | | | |
| Bacterioferritin | Iron storage | bfr | 18 | 18.4 | A0A4V3X737 | 50 |
| ATP synthase subunit | Produces ATP | atpA | 58.4 | 57.7 | C1AW01 | 20 |
| alpha | from ADP across | | | | | |
| | the membrane | | | | | |
| Citrate synthase | Involves in the | gltA | 39.69 | 39.2 | F8QR44 | 20 |
| | pathway | | | | | |
| | tricarboxylic acid | | | | | |
| | cycle | | | | | |
| 50S ribosomal protein | Ribonucleoprotein | bipA | 67 | 66.9 | A0A1G5MQC5 | 5 |
| L3 | | | | | | |
| ATP synthase subunit | Produces ATP | atpD | 49.5 | 50 | A0A024ED61 | 5 |
| beta | from ADP in the | | | | | |
| | presence of a | | | | | |
| | proton gradient | | | | | |
| | across the | | | | | |
| | membrane | | | | | |
| Glutamine synthetase | Glutamate- | glnA | 51.6 | 51.6 | A0A5D3GGF0 | 5 |
| | ammonia ligase | | | | | |
| | activity | | | | | |
| Elongation factor Tu | Promotes the | tuf | 43.57 | 43.2 | A0A1H2I8D2 | 5 |
| | GTP-dependent | | | | | |
| | binding of | | | | | |
| | aminoacyl-tRNA | | | | | |
| Alkyl hydroperoxide | Plays a role in cell | OU5_0185 | 20.4 | 20.8 | A0A024E2Y4 | 5 |
| reductase C | protection against | | | | | |
| | oxidative stress | | | | | |
| | by detoxifying | | | | | |
| | peroxides | | | | | |
| Alkyl hydroperoxide | response to | ahpF | 55.8 | 54.9 | K9NK88 | 4 |
| reductase subunit F | reactive oxygen | | | | | |
| | species | | | | | |
| 60 kDa chaperonin | Prevents | groL | 56.6 | 56 | B0KFQ2 | 3 |
| | misfolding and | | | | | |
| | promotes the re- | | | | | |
| | folding and proper | | | | | |
| | assembly of | | | | | |
| | unfolded | | | | | |
| | polypeptides | | | | | |
| | produced under | | | | | |
| | stress conditions | | | | | |
| Chaperone protein | Acts as a | dnak | 65.73 | 65.7 | A0A161TLX0 | 3 |
| Dnak | chaperone. | | | | | |
| DNA-directed RNA | Catalyzes the | rpoC | 154.9 | 154.5 | A0A4S4IZF1 | 2 |
| polymerase subunit | transcription of | | | | | |
| beta | DNA into RNA | | | | | |
| Polyribonucleotide | Involves in mRNA | pnp | 74.9 | 75.3 | A0A0K8M9A6 | 2 |
| nucleotidyltransferase | degradation | | | | | |
| Protein translocase | Has a central role | secA | 102.9 | 102.9 | BOKFR8 | 1 |
| subunit SecA | in coupling the | | | | | |
| | hydrolysis of ATP | | | | | |
| | to the transfer of | | | | | |
| | proteins into and | | | | | |
| | across the cell | | | | | |
| | membrane | | | | | |
| Aconitate hydratase | Involves in | acnA | 100.4 | 100.1 | A0A1XOUMG3 | 1 |
| | tricarboxylic acid | | | | | |
| | cycle | | | | | |

¹Molecular mass was calculated from the respective UniprotKB entry (Theoretical Mass).
²Molecular mass was obtained from LC-MS/MS (Experimental Mass).

Several proteins that are associated with oxidative stress, which prevent the unfolding of proteins were detected. High levels of reactive oxygen or chlorine species (ROS/RCS) cause protein unfolding and aggregation in the cytosol. To cope with this stress, bacterial cells employ ATP-independent chaperones such as the DnaK system and the GroEL system, which bind unfolded or aggregated proteins and maintain their solubility¹⁹. It is reported that the oxidation of PAH trans-dihydrodiols to catechols generates reactive ROS.²⁰. The presence of chaperones in the crude cocktail obtained from mixed-culture of PAH-degrading bacteria indicated that growth on these compounds induces a stress response. Similarly, Tomas-Gallardo et al., (2006) reported that Rhodococcus sp. strain TFB produced 60-kDa Chaperonins in the presence of phthalate. They identified phthalate-induced proteins using matrix-assisted laser desorption/ionization mass spectrometry (MALDI-MS/MS)²¹.

### Effect of Environmental Factors on Enzyme Activity

Six different temperatures including 10, 15, 20, 30, 40, and 50° C. and seven different pH values including 3, 4, 5, 6, 7, 8, and 9 were tested for enzyme activity assays. For the salinity test, five solutions of enzyme mixture were mixed with different NaCl concentrations ranging from 2 to 10% (w/v). The preferred temperature and pH values for the enzyme mixture are 20° C. and 7 respectively. Salt concentrations up to 5% have no effect on enzyme activities.

### Example 2: Application of Enzyme Mixture in Soil

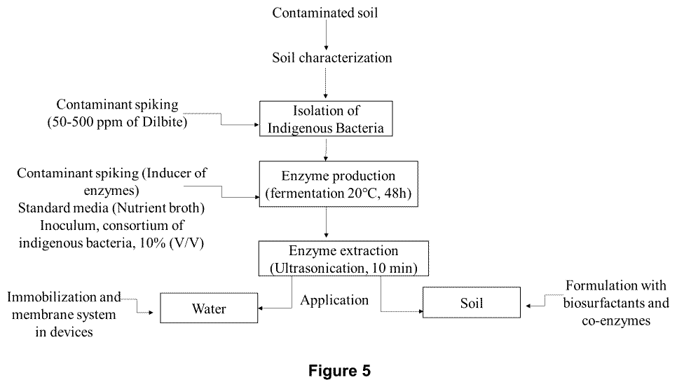

This procedure is generally outlined in FIG. 5.

### Laboratory Experiments Approach for Isolation and Identification of PAH Degrading Bacteria from Contaminated Sediments

Briefly, 0.5 g of the impacted sediments was transferred in a crimp sealed serum bottle (150 mL) containing 25 mL of MSM supplemented with 200 mg/L of oil as the sole carbon source and incubated for a month at 4° C. After 1 month of incubation of the final enrichment culture on the mixture of oils, aliquots (100 μl) of 10⁻¹⁰ dilutions, in MSM were spread on agar plates (M3 and NB agar) through the conventional spread-plate technique and then incubated at 15° C. for 4 days. After that, single colonies with different morphology were selected and each of them inoculated to the freshly prepared MSM or NB medium (50 mL) and used as inoculum for degradation and enzyme production screening^{7,22}.

To investigate the ability of individual strains to degrade PAHs at 15±1° C., 6×10⁹ CFU/mL (exponential phase bacteria) of each bacterial cell were spiked to each sample. All tests were performed in MSM and PAHs (naphthalene, anthracene, phenanthrene, and pyrene) as the sole carbon sources. To control the freely dissolved concentration of PAHs rather than the total concentration, the PAHs were loaded on the silicone O-rings before culturing the bacteria as described in the next section. The mixture was then incubated at 15±1° C. on a rotary shaker at 150 rpm. These conditions were selected based on optimum growth and aerobic PAH biodegradation using the above-mentioned genera ²³. The biodegradation efficiency was calculated by the following Eq. (1)

$$Degradation ( \% ) = \frac{{c}_{n} - {c}_{o}}{{c}_{o}} \times 100 \qquad ( 1 )$$

where, Cₒ and Cₙ are the concentration of test and abiotic control, respectively. Among a total of 18 isolates obtained, four isolates were selected based on their ability to grow on PAHs in separate and mixture substrate tests at 15±1° C. Bacterial growth was measured using optical density (OD) with a spectrophotometer at A600 nm (OD600 nm˜1-1.2 that corresponded to 1.1×109 cells/ml).

Finally, the selected colonies were identified with 16S ribosomal RNA gene, the gene fragment was amplified using the following universal primers for PCR: 27F (5′-AGAGTTTGATCCTGGCTCAG-3′), 1492R (5′-GGTTACCTTGTTACGACTT-3′). The amplified products were sequenced and then the 16S rRNA fragment sequences were compared with the reference sequences using the BLASTn program) in the National Center for Biotechnology Information (NCBI) database²⁴. Thereafter, the phylogenetic analysis was developed using the neighbor-joining algorithm of MEGA 7.0 software with 1000 bootstraps²⁵.

### Laboratory Experiments Approach for In-Situ Soil Application

For soil application, enzymatic biodegradation in soil involves key factors that should be verified in batch tests and soil columns. Key factors are enzyme stability, degradation kinetics, dispersion of enzymes, and availability of contaminants. The adsorption of enzymes/proteins on the soil solid phase is known as a quasi-reversible phenomenon with great significance. Adsorption can stabilize enzymes by increasing their resistance toward denaturation. However, the adsorption of contaminants can reduce the bioavailability of the substrate for the enzymatic reaction. Dispersing aids such as surfactants, biosurfactants can be added to the enzyme mixture.

### Soil Characteristics

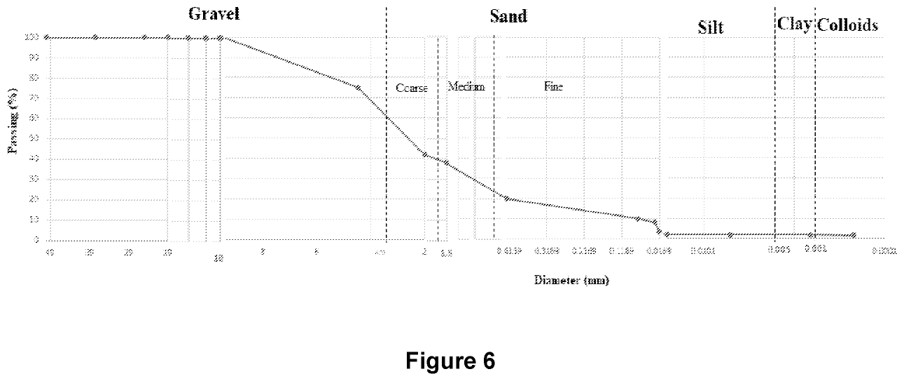

The soil samples used for column test experiments have been collected from a contaminated site in Quebec, Canada. The initial content of metals is reported in Table 6 which is applicable norms in Quebec for commercial and industrial uses. The soil samples were originally contaminated with PAHs (Table 3) and have been artificially contaminated until an initial concentration of 1000 μg/kg for each target PAHs (1:1:1:1) was obtained (average concentration in site). Sieving analysis was carried out according to the ASTM D422-63 procedure (ASTM 2007). Grain size distribution and characteristics of the soil are described in Table 4. Particle-size analysis of the soil is shown in FIG. 6.

TABLE 3
Physico-chemical properties of the soil from a contaminated site.

| Physico-chemical properties | | |
|---|---|---|
| pH | | 6.5 ± 0.5 |
| Porosity | | 0.29 ± 0.08 |
| Moisture content | | 13.6 ± 0.5 (% dry weight) |
| Water holding capacity | | 71 ± 2 (%) |
| Total carbon | | 2.6 ± 0.3 (%) |
| Nitrogen | | 0.2 ± 0.1 (%) |
| Soil type | Gravel | 25.0 (%) |
| | Sand | 64.8 (%) |
| | Silt | 8.3 (%) |
| | Clay | 0.1 (%) |
| | Colloids | 1.8 (%) |

TABLE 4
Size analysis of soil (Standard method form ²⁶)

| | Diameter (mm) | | | | | | | | | | | | | | |
|---|---|---|---|---|---|---|---|---|---|---|---|---|---|---|---|
| | 50 | 37.5 | 25 | 19 | 9.5 | 4.75 | 2 | 0.425 | 0.075 | 0.0289 | 0.0169 | 0.0121 | 0.0086 | 00.42 | 0.0018 |
| Passing | 100 | 100 | 100 | 100 | 100 | 75 | 42 | 20 | 10.2 | 8.4 | 3.6 | 2.2 | 1.9 | 1.8 | 1.7 |
| (%) | | | | | | | | | | | | | | | |

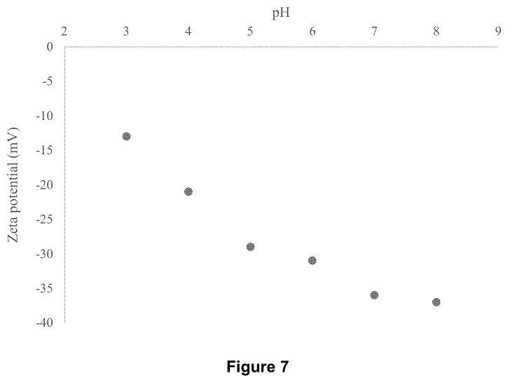

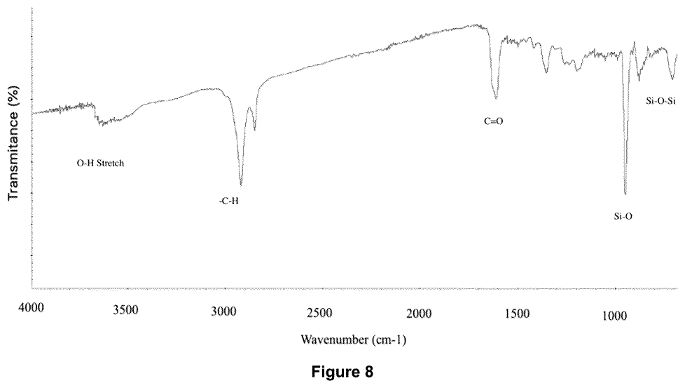

Basic characteristics of the soil, such as zeta potential (FIG. 7), and FTIR analysis (FIG. 8) were determined by standard methods. The surface charge of soil particles was studied using zeta-sizer Nano ZS (Malvern Instrument Inc., UK). The Fourier transform infrared (FT-IR) spectroscopic analyses were recorded in the range of 400-4000 cm⁻¹ using a Nicole IS50 FT-IR Spectrometer (Thermo Scientific, USA) in attenuated total reflectance (ATR) mode with 8 cm⁻¹ resolution as reported elsewhere.

### Soil Column Set-Up

The soil columns used were made from a stainless-steel cylinder (wall thickness of 2 mm, 3.5 cm in diameter, and 14.5 cm in length (140 mL)). Each set had two replicates. Tubes were clamped between Teflon end reservoirs and stainless-steel endplates. To avoid particle losses, one stainless-steel mesh was inserted at both the inlet and outlet ends of the column, and two Vitton© O-rings were used to seal the cylinder with the endplates. Column packing and water saturation were carried out according to Martel and Gelinas (²⁷. Briefly, dry packing was carried out by pouring discrete soil portions (with a spoon) into the column and then mechanically compacting them with a plunger. The soil portions were deposited in layers of 1 cm and each layer was artificially contaminated with selected polyaromatic hydrocarbons using a micropipette of 100 μL to a final detected concentration in soil. To ensure hydraulic connectivity between the layers, lightly scarifying the soil surface (0.5 cm of each layer) was carried out after compaction and before the addition of another layer. This procedure was repeated several times until the top of the soil column was reached (Gilbert et al. 2014). The bulk density of the soil after packing was 1.8±0.02 g/cm³. The columns were saturated with water overnight, following the injection of CO₂ at a pressure of 4 psi to remove entrapped air for complete water saturation. As CO₂ is a water-soluble gas, its bubbles can quickly dissolve in water. The soil columns were allowed to saturate with deaerated water (approximate 5 pore volume) using a peristaltic pump (2 mL/min) from bottom to top to eliminate CO₂. Polyethylene tubes were used for the inflow and outflow of the aqueous solutions.

### Soil Column Characterization

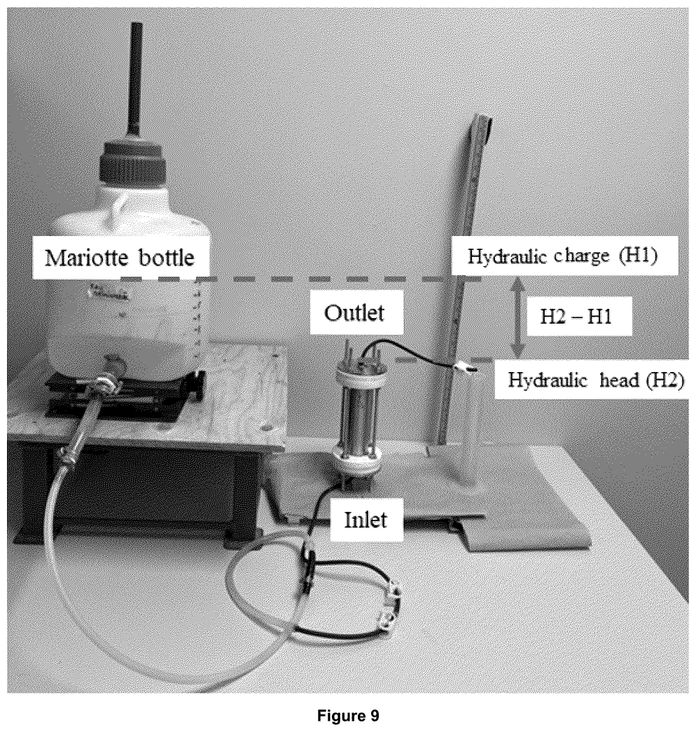

For laminar flow through the porous column bed, the hydraulic conductivity K of each column was calculated using Darcy's law (Eq. (2)) following a permeability test which was conducted in duplicates. Three hydraulic gradients were imposed for each column at 5, 10, and 15 cm/cm (FIG. 9).

This range of hydraulic gradients might be appropriate for the packed column because it did not alter soil fabric, structure, and the rearrangement of the soil particles and it did not result in channelization in the cracks in the soil samples (Mohamed and Paleologos 2017).

$$K = \frac{Q}{A \times \frac{( H 2 - H1 )}{L}} \qquad ( 2 )$$

Where K is hydraulic conductivity (cm/s), Q is the flux (cm³/s), A is cross-sectional area (cm²) H₂ and H₁ are the hydraulic heads and L is the length of column (cm). Hydraulic conductivity, indicating permeability of porous media, measurement is necessary to study the ability of fluid (enzyme mixture in aqueous solution) to flow through the porous media and evaluate the enzyme accessibility to contaminated soils. The pore volume of each column was measured according to Eq. (3):

PV=m₁−m₂−m₃ (3)

Where, m₁, m₂ and m₃ are the saturated soil column mass, the mass of water in both ends reservoir (including the attached tubing sections at the time of water injection) and the mass of the dry column respectively. The conversion of the mass of water into volume was assumed at room temperature to calculate the pore volume. The pore volume and the porosity (the media's own internal porosity (pore volume/total volume of the soil column) of each packed column were evaluated^{28, 29}.

The dynamic behavior of water flowing into the packed soil columns was described by measuring the axial dispersion coefficient to evaluate the assumption of dispersed plug flow. The axial dispersion coefficient (D_{L}) in the packed column was measured by injecting a conservative tracer (Br⁻). This ion is not initially present in soil and in enzyme solution, is not affected by soil adsorption, and has no effect on enzymes activity. The concentration of Br⁻ collected in the effluent samples over time was measured using an ion-selective electrode detector. Dispersity by using the one-dimensional Ogata-Banks (1-D) dispersion according to Eq. (4):

$$\frac{C}{{C}_{0}} = \frac{1}{2} [ erfc ( \frac{x - vt}{\sqrt{4 {D}_{L} t}} ) ] \qquad ( 4 )$$

Where, C is the concentration in column outflow, C₀ is the initial Br concentration (1000 μg/L in this study), erfc is the complementary error function, x is the depth (position in the column), v is the pore water velocity, and t is the time. Dispersion coefficient measurement is needed to evaluate if the column is packed properly and if a diffusion-dominated flow may exist. The volume of Br solution injected at C/C₀=0.5 gives the transport pore volume of the column. The ratio of the volume of enzyme solution injected at C/C₀=0.5 to the one of Br gives the retardation factor (R_{enzyme}) or the adsorption coefficient (K_{D}) of the enzyme in the soil according to Eq (5) and (6). This is very important information to calculate how much enzyme solution is needed to flood completely the porous media while achieving an enzyme activity that is equivalent to the solution being injected. Fetter et al. (1999) showed that the Freundlich sorption isotherm coefficient (K_{f}) can be calculated using the retardation factor as follow

$${K}_{f} = \frac{( {R}_{e n z y m e} - 1 ) θ}{n {C}^{n - 1} {ρ}_{b}} \qquad ( 5 )$$

$${R}_{enzyme} = {V}_{enzyme} / {V}_{{Br}^{-}} \qquad ( 6 )$$

Where R is the retardation factor, θ the porosity, ρ_{b} the bulk density and n Freundlich isotherm constant. (Table 5).

TABLE 5
Characterization and initial condition of the soil column experiment

| Soil | Column | Soil density in | Soil intrinsic | Pore | Hydraulic |
|---|---|---|---|---|---|
| column | volume | column ⁽¹⁾ | permeability | volume | conductivity |
| No. | (cm³) | (g/cm³) | (m²) | (cm³) | (K_{w}) |
| 1 | 140.39 | 1.88 | 7.5-9.2 × 10⁻¹³ | 50 ± 0.5 | 1.3 × 10⁻⁶ |
| 2 | 139.37 | 1.79 | | 49 ± 0.5 | 1.4 × 10⁻⁶ |
| 3 | 141.41 | 1.71 | | 50 ± 0.5 | 2.4 × 10⁻⁶ |
| 4 | 140.39 | 1.72 | | 50 ± 0.5 | 2.2 × 10⁻⁶ |
| 5 | 139.36 | 1.82 | | 49 ± 0.5 | 1.5 × 10⁻⁶ |
| 6 | 140.39 | 1.78 | | 50 ± 0.5 | 2.3 × 10⁻⁶ |
| 7 | 139.36 | 1.90 | | 49 ± 0.5 | 2.4 × 10⁻⁶ |
| 8 | 139.36 | 1.84 | | 50 ± 0.5 | 2.5 × 10⁻⁶ |
| 9 | 139.36 | 1.90 | | 50 ± 0.5 | 1.6 × 10⁻⁶ |
| 10 | 139.36 | 1.81 | | 49 ± 0.5 | 2.9 × 10⁻⁶ |
| 11 | 140.39 | 1.70 | | 50 ± 0.5 | 1.7 × 10⁻⁶ |

⁽¹⁾ Soil density was selected based on site soil density ranging 1.7-1.9 g/cm³.

### Application of Enzyme Mixture in Soil

To design the best-formulated enzymes for biodegradation of specific contaminates, site characterizations such as pH, annual temperature, contaminant concentration, salinity, metal concentration are needed. For example, the contaminated soils used in this study are collected from a contaminated site in Quebec, Canada. This site is selected because of its intense oil contamination which contained ˜1,000 mg/kg of crude oil in the soil. The initial characterizations of the samples are presented in Table 6.

TABLE 6
Physico-chemical properties of the soil of the contaminated site.

| Physico-chemical properties | | Content (μg/Kg) |
|---|---|---|
| Polycyclic Aromatic | Naphthalene | 1120 ± 104.24 |
| Hydrocarbons | Anthracene | 983 ± 198.6 |
| | phenanthrene | 1040 ± 106.8 |
| | Pyrene | 820 ± 251.6 |
| Metals | Iron (Fe) | 10812.66 ± 44.06 |
| | Copper (Cu) | 14.26 ± 0.60 |
| | Chromium (Cr) | 105.02 ± 6.10 |
| | Cadmium (Cd) | 0.65 ± 0.26 |
| | Aluminum (Al) | 1018.45 ± 10.80 |
| | Manganese (Mn) | 255.26 ± 7.20 |
| | Nickel (Ni) | 104.08 ± 1.00 |
| | Arsenic (As) | 6.42 ± 0.70 |
| | Lead (Pb) | 4.99 ± 0.20 |
| | Zinc (Zn) | 40.17 ± 2.80 |

| | |
|---|---|
| pH | 7.1 ± 0.5 |
| Average Annual Temperature | 15 ± 2 (° C.) |
| Salinity | |
| Moisture content | 13.6 ± 0.5 (% dry weight) |
| Water holding capacity | 75 ± 2 (%) |

### Remediation Goals

Risk assessment can help to determine goals of remedial action at a site. The maximum level of residual risks that remain at the site after clean-up needs to be specified. The concentration value of the target contaminant is referred to as a preliminary remediation goal. The preliminary remediation goal is the average concentration of target chemicals in an exposure area that will yield the specified target risk in an individual who is exposed at random within the exposure area. The average concentration after remediation is required to be reduced to the preliminary remediation goal or below. Since the contaminated site is located in the province of Quebec (Canada), total PAHs 0.1 mg/kg of soil is considered as the local soil threshold and a benchmark for evaluating the efficiency of treatment³⁰.

### Batch Biodegradation Tests in Soil

The contaminated soil and groundwater were provided from a petroleum site with intense contamination of oil. The mineralogy of soils varies in different parts of the site but, in addition to quartz and feldspar, clay minerals and carbonates were also found. The results of groundwater characterization also confirmed the presence of carbonates (hard water) with a pH of 7.2±0.4. The soil sample comprised 59% of particles with a size range between 1-5 mm, 38% with a size range of 250 μm-500 μm and 3% of very fine particles (less than 250 pm in size). The physical characteristics of the soil were determined as follows: moisture content of 24.17±0.5%, pH 7.1±0.2, total solids: 75.36±0.91%. Batch tests indicated that 85-90% of PAHs were degraded after 4 weeks (Table 7).

TABLE 7
Batch test PAHs biodegradation using enzyme solution in soil

| | Initial | concentration | |
|---|---|---|---|
| | concentration | (μg/Kg) | Bio- |
| PAHs in soil | (μg/Kg) | after 4 weeks | degradation (%) |
| Naphthalene | 1120 ± 104.24 | 210.5 ± 53.2 | 81.25 |
| Anthracene | 983 ± 198.6 | 256.8 ± 21.56 | 73.8 |
| phenanthrene | 1040 ± 106.8 | 310 ± 31.04 | 70.19 |
| Pyrene | 820 ± 251.6 | 207 ± 56.08 | 74.75 |

### Enzymatic Biodegradation of PAHs in Soil Columns

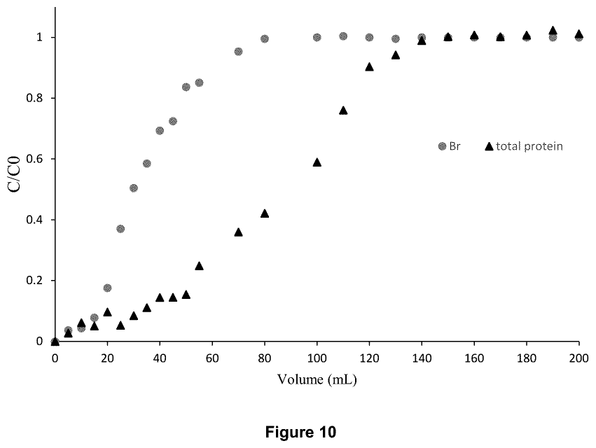

The packed soil columns include six treatments are shown in Table 8. The enzyme mixture contained around 30 U/mg of naphthalene dioxygenase, 40 U/mg of dihydrodiol dehydrogenase, and 40 U/mg of catechol-2,3-dioxygenase, 10 U/mg of salicylaldehyde dehydrogenase, 10U/mg of salicylate hydroxylase and 5 U/mg of Trans-2-carboxybenzalpyruvate hydratase-aldolase was injected into the soil column as influents until saturation. The effluents were collected for each 5 mL at regular intervals to determine the total protein concentration and Br concentration (FIG. 10).

TABLE 8
Soil column treatment and experimental conditions.

| Soil | | | |
|---|---|---|---|
| column | Experimental | Treatment | |
| No. | properties | duration | Description |
| 1 | Autoclaved soil | 4 weeks | As a sterile control for |
| 2 | column | 8 weeks | determination of natural |
| | | | biodegradation rate |
| 3 | Untreated soil | Time 0 | As a control for |
| 4 | column | 4 weeks | determination of the |
| 5 | | 8 weeks | initial concentration of |
| | | | contaminant and natural |
| | | | biodegradation rate |
| 6 | | 4 weeks | |
| 7 | Treated soil column | 8 weeks (with an | X corresponds to the |
| | with enzyme | enzyme injection | enzyme mixture without |
| | concentration X | after 4 weeks) | dilution |
| 8 | Treated soil column | 4 weeks | X/5 corresponds to 5 |
| 9 | with enzyme | 8 weeks (with an | times dilution of the |
| | concentration X/5 | enzyme injection | enzyme mixture |
| | | after 4 weeks) | |
| 10 | Treated soil column | 4 weeks | X/10 corresponds to 10 |
| 11 | with enzyme | 8 weeks (with an | times dilution of the |
| | concentration X/10 | enzyme injection | enzyme mixture |
| | | after 4 weeks) | |

As mentioned in Table 9, the soil column tests were carried out over 8 weeks at 15±1° C. (average annual temperature of the targeted site). The enzyme injections were repeated after 4 weeks for the columns that were planned to open after 8 weeks. The time intervals for injection were selected based on the enzyme stability results in batch tests.

To determine the PAHs concentration in soil samples, about 0.1 g of soil were taken from the head, middle and bottom part of the soil column (depth of 2, 7, and 12 cm) in triplicate for each part. Each sample was homogenized, and a 5 g subsample was placed in a 43 ml vial where it was mixed with 5 mL of hexane as mentioned in PAH concentration analysis. The results showed that 50-70% of PAHs were degraded after 4 weeks (Table 9).

TABLE 9
PAH concentration in soil samples from soil columns in time 0 and after 4 weeks
(4 W) and 8 weeks (8 W) of incubation. Each sample was analyzed in triplicate.

| Soil | | | | | | |
|---|---|---|---|---|---|---|
| column | | Concentration (μg/Kg) | | | | Biodegradation |
| No. | Description | Naphthalene | Anthracene | phenanthrene | Pyrene | (%) of total PAH |
| 1 | Autoclaved soil | 1340 ± | 1040 ± | 1156 ± | 845 ± | — |
| | column (4 W) | 162 ^{a} | 54 ^{a} | 31 ^{c} | 17 ^{a} | |
| | | 1061 ± | 1272 ± | 1907 ± | 915 ± | |
| | | 80 ^{b} | 34 ^{b} | 91 ^{b} | 12 ^{b} | |
| | | 1100 ± | 1203 ± | 1100 ± | 1098 ± | |
| | | 74 ^{c} | 52 ^{c} | 92 ^{c} | 32 ^{c} | |
| 2 | Autoclaved soil | 1255 ± | 1007 ± | 1105 ± | 1205 ± | — |
| | column (8 W) | 51 ^{a} | 33 ^{a} | 72 ^{a} | 87 ^{a} | |
| | | 907 ± | 1201 ± | 1080 ± | 1080 ± | |
| | | 17 ^{b} | 14 ^{b} | 61 ^{b} | 76 ^{b} | |
| | | 600 ± | 1190 ± | 992 ± | 1009 ± | |
| | | 62 ^{c} | 46 ^{c} | 66 ^{c} | 57 ^{c} | |
| 3 | Untreated soil | 1101 ± | 1203 ± | 1040 ± | 1120 ± | — |
| | column (time 0) | 224 ^{a} | 198 ^{a} | 106.8 ^{a} | 251 ^{a} | |
| | | 1100 ± | 1201 ± | 1060 ± | 1050 ± | |
| | | 27 ^{b} | 54 ^{b} | 15 ^{b} | 44 ^{b} | |
| | | 1005 ± | 1150 ± | 1015 ± | 1065 ± | |
| | | 43 ^{c} | 102 ^{c} | 31 ^{c} | 31 ^{c} | |
| 4 | Untreated soil | 1023 ± | 1351 ± | 967 ± | 1086 ± | 3.28 |
| | column (4 W) | 144 ^{a} | 110 ^{a} | 61 ^{a} | 46 ^{a} | |
| | | 1023 ± | 1200 ± | 985 ± | 1010 ± | |
| | | 73 ^{b} | 54 ^{b} | 44 ^{b} | 85 ^{b} | |
| | | 935 ± | 1205 ± | 930 ± | 1033 ± | |
| | | 65 ^{c} | 51 ^{c} | 302 ^{c} | 50 ^{c} | |
| 5 | Untreated soil | 992 ± | 1907 ± | 967 ± | 1053 ± | 3.41 |
| | column (8 W) | 31 ^{a} | 91 ^{a} | 61 ^{a} | 32 ^{a} | |
| | | 923 ± | 1100 ± | 985 ± | 979 ± | |
| | | 23 ^{b} | 62 ^{b} | 44 ^{b} | 70 ^{b} | |
| | | 906 ± | 1200 ± | 930 ± | 1002 ± | |
| | | 95 ^{c} | 74 ^{c} | 32 ^{c} | 14 ^{c} | |
| 6 | Treated soil | 634 ± | 505 ± | 617 ± | 673 ± | 36.75 |
| | column with | 18 ^{a} | 37 ^{a} | 18 ^{a} | 92 ^{a} | |
| | enzyme | 590 ± | 665 ± | 985 ± | 629 ± | |
| | concentration X | 72 ^{b} | 82 ^{b} | 44 ^{b} | 56 ^{b} | |
| | (4 W) | 579 ± | 680 ± | 630 ± | 641 ± | |
| | | 48 ^{c} | 32 ^{c} | 40 ^{c} | 24 ^{c} | |
| 7 | Treated soil | 466 ± | 605 ± | 489 ± | 313 ± | 53.21 |
| | column with | 89 ^{a} | 17 ^{a} | 29 ^{a} | 31 ^{a} | |
| | enzyme | 433 ± | 380 ± | 462 ± | 495 ± | |
| | concentration X | 23 ^{b} | 46 ^{b} | 59 ^{b} | 61 ^{b} | |
| | (8 W) | 472 ± | 490 ± | 496 ± | 504 ± | |
| | | 13 ^{c} | 16 ^{c} | 11 ^{c} | 27 ^{c} | |
| 8 | Treated soil | 746 ± | 729 ± | 707 ± | 826 ± | 27.48 |
| | column with | 79 ^{a} | 7 ^{a} | 19 ^{a} | 36 ^{a} | |
| | enzyme | 723 ± | 855 ± | 715 ± | 747 ± | |
| | concentration | 82 ^{b} | 12 ^{b} | 50 ^{b} | 54 ^{b} | |
| | X/5 (4 W) | 685 ± | 712 ± | 688 ± | 764 ± | |
| | | 51 ^{c} | 21 ^{c} | 20 ^{c} | 43 ^{c} | |
| 9 | Treated soil | 486 ± | 125 ± | 473 ± | 515 ± | 51.9 |
| | column with | 81 ^{a} | 41 ^{a} | 82 ^{a} | 79 ^{a} | |
| | enzyme | 452 ± | 65 ± | 482 ± | 479 ± | |
| | concentration | 27 ^{b} | 2 ^{b} | 65 ^{b} | 71 ^{b} | |
| | X/5 (8 W) | 389 ± | 85 ± | 455 ± | 490 ± | |
| | | 58 ^{c} | 1 ^{c} | 27 ^{c} | 89 ^{c} | |
| 10 | Treated soil | 953 ± | 812 ± | 937 ± | 1009 ± | 3.85 |
| | column with | 39 ^{a} | 11 ^{a} | 19 ^{a} | 69 ^{a} | |
| | enzyme | 993 ± | 920 ± | 955 ± | 950 ± | |
| | concentration | 31 ^{b} | 12 ^{b} | 45 ^{b} | 51 ^{b} | |
| | X/10 (4 W) | 906 ± | 981 ± | 867 ± | 971 ± | |
| | | 59 ^{c} | 82 ^{c} | 20 ^{c} | 23 ^{c} | |
| 11 | Treated soil | 943 ± | 889 ± | 917 ± | 1003 ± | 15.55 |
| | column with | 34 ^{a} | 72 ^{a} | 56 ^{a} | 35 ^{a} | |
| | enzyme | 876 ± | 910 ± | 935 ± | 930 ± | |
| | concentration | 38 ^{b} | 16 ^{b} | 31 ^{b} | 51 ^{b} | |
| | X/10 (8 W) | 860 ± | 969 ± | 880 ± | 851 ± | |
| | | 70 ^{c} | 2 ^{c} | 15 ^{c} | 27 ^{c} | |

Samples from
^{a} head (depth of 1-2 cm),
^{b} middle *dept of ((depth of 7-8 cm) and
^{c} bottom (depth of 12-13 cm) of soil column.

### Example 3: Application of Enzymes for Marine environment

### Laboratory Experiments Approach for Marine Application

Laboratory Experiments Approach for Preparation and Characterization of the Jellyfish-Like Device

### Selection and Characterization of Hollow Fiber Membranes for Circulation of Enzymes

Spectrum® modified poly(ether sulfone) mPES hollow fibers with a molecular weight of (MWCO) of 10,000 Da (D02-E010-05-N, Spectrum Labs, USA) were used for multiple enzyme and cofactor immobilization. Since the molecular weight of PAH substrates is about 169 and the molecular weight of the studied enzyme is more than 10,000 Da, Spectrum® mPES membrane appeared appropriate for this study. Moreover, thin-skinned hydrophilic modified polyether sulfone (mPES) hollow fibers are void-free and anisotropic in a structure that provides high relative hydraulic permeability and flux rates at a maximum pressure of 30 psi. Hydrophilic neutrally charged Spectrum® mPES hollow fiber minimized non-specific protein binding as well as fouling by hydrophobic molecules³¹. The hollow fiber ultrafiltration module consisted of 10 fibers with an internal diameter of 1.0 mm, a length of 45 cm, a wall thickness of 0.1 mm, and the total surface area was 58 cm².

After selecting the membrane, the second step was to ensure that these membrane modules were intact. To do so, the diffusion test method was applied to evaluate its integrity.

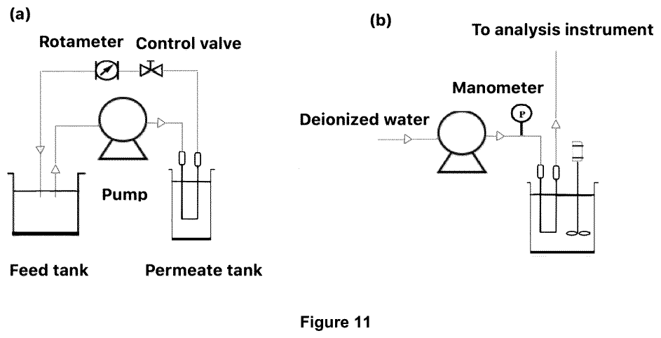

The second step was the systematic exploration of membrane hollow-fibers characteristics including the permeation of substrate and the retainment ratio of the co-enzymes. The permeation equipment, shown schematically in FIG. 11, consisted of high-pressure liquid pumps, the analytical instrument including a UV-visible spectrophotometer, was applied to determine retention (R) of co-enzymes (NAD⁺) in continuous mode. The effect of salinity and polyethyleneimine (PEI) on the NAD⁺ retention ratio was also studied to determine the optimum condition for physical entrapment of co-enzyme within the membrane. The main advantage of using PEI and enhancement of retainment ratio (R-value) for the coenzymes is that the kinetics of the enzymes does not change.³²

Recycling-pump speed was fixed at 50 ml min⁻¹ to circulate enzyme solution or co-enzyme (NADH) in 20 mM Tris-HCl buffer pH=9. The retention of 1 mM enzyme or co-enzyme (NADH) inside the lumen of hollow fiber membrane was studied by calculating the retainment ratio as follow

$$R = \frac{ln \frac{C}{{C}_{0}}}{ln \frac{{V}_{O}}{V}} \qquad ( 7 )$$

Where V, V₀ C, and C₀ are the volumes of the inner solution before and after the permeation experiments and the solute (i.e., coenzyme) concretions at time t, and t₀. The concentration of co-enzymes (NAD⁺) retained in pre-selected ultrafiltration with 10 kDa were measured via Biowave UV-vis spectrophotometer at 260 nm, respectively.

To determine the permeation coefficient for substrates, single fiber reactor consisting of a gas-liquid chromatograph was applied. Experimental data were analyzed according to the mass exchanger equation (Eq. (8)):³³

$$ln ( 1 - \frac{{C}_{I ( z = 0 )}}{{C}_{2} ( z = l )} ) = [ \frac{- 1}{\frac{1}{{K}_{0}} + \frac{a + b}{b {k}_{i}} \times \frac{{( a + b )}^{ln \frac{a + b}{a a}}}{{k}_{i 2} {D}_{i 2}}} ] \times \frac{1}{Q} \qquad ( 8 )$$

Where K₀, Kᵢ, a, a+b, Dᵢ₂ and Kᵢ₂ are mass transfer coefficients for the external flowing stream, mass transfer coefficient for the internal flowing stream, the radius of the hollow region in fiber (I.D), the diffusion coefficient of permeating solute in fiber wall and solubility of permeating solute in fiber wall respectively. The high flow rate of the internal flowing stream (100 ml/min) and vigorous agitation at 500 in a 1L flask were adjusted so that the first and second terms in the denominator of the quantity inside the brackets were small (the absence of internal and external diffusional limitation). By assuming the high mass-transfer coefficient for the external and internal flowing streams, the permeation coefficient of the substrate can be calculated (Pᵢ₂=kᵢ₂Dᵢ₂).

### Application of the Jellyfish-Like Device

To study the benefit of using hollow fiber modules for enzyme-catalyzed reactions, the performance of a beaker-type hollow fiber system was evaluated theoretically based on simple mathematical models and then examined experimentally in the degradation of the model substrate. Ultrafiltration oscillation is induced in the beaker side of the reactor by introducing a pulsatile flow to the hollow fiber lumen as well as by maintaining outflow from the lumen at the average value of the pulsatile inflow rate. The phenomenological transport equation may be written according to Kedem and Katachalsky³⁴

Jₛ=PₘΔC+σJ_{w}C_{b} (9)

Where σ, C_{b}, ΔC, Jₛ, and J_{w} refer to sieving coefficient, solute concertation in the bulk solution phase, concentration difference between the two compartments, substrate, and water flux, respectively. The sieving coefficient

$$( σ = \frac{{C}_{filtrate}}{{C}_{b}} )$$

in the absence of concentration polarization can be 1³⁵ Mass balance for the substrate in compartments I and II, respectively, are

$$\frac{d ( {V}_{I} {S}_{I} )}{dt} = - {AJ}_{s} + Q ( {S}_{I 0} - {S}_{I} ) \qquad ( 10 )$$

$$\frac{d ( {V}_{II} {S}_{II} )}{dt} = A {J}_{s} - {V}_{II} \frac{{V}_{max} {S}_{II}}{{k}_{m} + {S}_{II}} \qquad ( 11 )$$

Where A and Q are mass transfer areas of hollow fiber membrane (cm²) and volumetric flowrate (cm³). If we assume square or sine wave ultrafiltration swing, in the absence of concentration polarization, water flux varies with time as

$${J}_{w} = [ \begin{matrix}{L}_{e} d g ( t ) & Square Wave \\ {L}_{e} d sin ( wt ) & Sine Wave\end{matrix}$$

Le d and d are the difference between the outlet and the inlet flowrates in the lumen appears as ultrafiltration and the fraction of average outlet velocity. For sine-wave ultrafiltration swing, Eq. (10) and (11) can be transformed into dimensionless forms by using the following dimensionless variables

$${S}_{1} = \frac{{S}_{I}}{{S}_{I 0}} τ = wt {\tilde{K}}_{m} = \frac{{K}_{m}}{{S}_{I O}} {λ}^{2} = \frac{{V}_{max} {V}_{II}}{{S}_{IO} {AP}_{m}} \partial = \frac{{V}_{II 0}}{{V}_{I 0}}$$

$$W = \frac{{wV}_{i o}}{A {P}_{m}} F = \frac{{V}_{i 0}}{{TAP}_{m}} Le = \frac{Q e}{{P}_{m} A} T = \frac{2 π}{W}$$

$${V}_{1} \frac{d ( {S}_{1} )}{d τ} - {L}_{e} d sin τ = - ( {S}_{1} - {S}_{2} ) - sin τ f ( {S}_{1} , {S}_{1} ) {L}_{e} d + L s ( 1 - {S}_{1} ) \qquad ( 12 )$$

$${V}_{2} \frac{d ( {S}_{2} )}{d τ} + {L}_{e} d sin τ = ( {S}_{1} - {S}_{1} ) + sin τ f ( {S}_{1} , {S}_{1} ) {L}_{e} d - \frac{{λ}^{2} {S}_{2}}{{\tilde{K}}_{m} + {S}_{2}} \qquad ( 13 )$$

$$Where {V}_{1} = \partial W - {L}_{e} d ( 1 - cos τ )$$

$${V}_{2} = \partial W + {L}_{e} d ( 1 - cos τ )$$

F and W are dimensionless frequency for square and sine wave pulsations. Eq. (12) and (13) were solved numerically using the Runge-Kutta scheme to study reactor performance with ultrafiltration swing.

### PAH concentration Analysis

The release of PAHs to water following the oil spill and its biodegradation in the presence of pre-selected strains and obtained enzymes solutions were confirmed by GC-MS (Agilent model 6890 GC, 5973 MSD) analyses which were carried out at 0, 30, 47 days of incubation.³⁶ Biotic and abiotic controls were used in the same conditions. The preparation of samples for GC/MS analysis was described elsewhere in detail.³⁶ The fermentative medium (20 mL) containing the remaining PAHs and metabolites and surrogate standards (Acenaphthene D10, Chrysene D12, Anthracene D10 each—0.7 μg·mL⁻¹, Pyrene D10—1.0 μg·mL⁻¹; purchased from Chromatographic Specialities Inc., Canada) were added to glass tubes (50 mL with covers). After mixing, 20 mL of Dichloromethane (HoneyWell™, Fisher Scientific, Canada) was added to each sample and mixed thoroughly for 30 minutes. 5 g of silica (SiliCycle, Canada) was added, and the tubes were shaken on an oscillating shaker for 10 minutes. 5 mL of supernatant were withdrawn and transferred in a calibrated glass tube, reduced to 1 mL (35° C. with nitrogen gas), and then analyzed by GC/MS (1 mL/min, 300° C., 35 minutes).³⁶.

### Silicone O-Ring Passive Dosing Format

It is very important to control the freely dissolved PAH concentration and keep it unchanged throughout the experiment by equilibrium partitioning from the silicone. Silicone O-rings (1 g each) were used as passive dosing format to provide constant freely dissolved concentrations and avoid spiking with cosolvents as described elsewhere.³⁷ In brief, the rings were loaded by equilibrium partitioning i.e., pushing PAHs (log K_{ow,}3.33-4.88) from a methanol suspension of naphthalene, anthracene, phenanthrene, pyrene into the polymer, followed by methanol removal with water.³⁸ The performance of passive dosing was determined by studying O-ring loading, substrate release into the medium and equilibrium concentration in the medium. During the loading of PAHs, individual PAHs were dissolved in methanol (loading solvent) to saturation level. To create the PAHs mixture, the concentration in the loading solution was calculated using partitioning coefficients for each PAH from Gilbert et. al. (2015).³⁹ Then, cleaned and dried O-rings were added to 50 mL volumes of loading solution kept at 15±1° C. A criterion for successful application of the passive dosing system was ensuring the mass of PAHs dissolved into methanol was much greater than that partitioned into the silicon O-ring which in turn was greater than that partitioned into the MSM³⁷. After 24, 72 and 128 h, O-ring was removed from each loading batch, and they were transferred into MSM to measure O-ring (Crest) and freely dissolved concentration of each PAH (C_{free}) at 0.33, 0.66, 1.33, 2.66, 5.33, 12, 24,48, and 72 h. The partition coefficient between different phases (K_{Methanol:Oring} and K_{Oring:MSM}) were obtained using the measured C_{test,initial} and C_{free, initial}. The obtained partitioning coefficient (K_{Oring:MSM}) was applied to calculate the required O-ring volume to avoid depletion (<5%) using the following Equation:

$${V}_{Oring} = \frac{{V}_{MSM}}{0.05 * {K}_{Oring : MSM}} \qquad ( 14 )$$

With a 5 mL MSM and partitioning coefficients from Table 10, the O-ring volume needed for each PAH and PAH mixture were determined. The initial concentrations of freely dissolved PAHs (C_{free initial}) and O-ring concentrations (Cₜₑₛₜ) in medium samples were calculated via the approach published by Butler et. al (2013).³⁷ Finally, for the determination of the losses, freely dissolved concentrations of PAHs were directly measured in medium samples to compare with calculated C_{free initial}.

Two controls were included to study biodegradation versus dissolved concentrations. The first control contained 5 mL of strain inoculating suspension in passive doing vials with O-rings to account for losses through volatilization. The second control contained 5 mL of MSM without any strain in passive dosing vials in the presence of O-ring loaded with PAHs to account for losses through biodegradation.

### Biodegradation versus Dissolved Concentration

Table 10 shows the partition coefficients obtained from measured PAH concentrations in loading solution, MSM, and O-ring. The losses of naphthalene, anthracene, phenanthrene, and pyrene through volatilization for control 1 were 19, 4, 0 and 0%, respectively. To buffer the losses, a 5-higher volume of O-ring was used for naphthalene to avoid depletion of more than 5%.

TABLE 10
Octanol-water coefficients (log Kow) and solubilities for PAHs
in MSM. Also dissolved concentrations measured in MSM as well
as the obtained partitioning coefficients for O-ring and MSM

| | | C_{MSM} (calculated) | C_{test initial} | C_{free initial} | C_{free} | K_{Oring:MSM} |
|---|---|---|---|---|---|---|
| | Log K_{ow} | mg/L | mg/L | mg/L | mg/L | mg/L |
| Naphthalene | 3.37 | 20.52 | 97.8 | 18.31 | 17.73 | 1402 ± 91 |
| Anthracene | 3.92 | 3.11 | 94.1 | 2.98 | 2.66 | 2559 ± 442 |
| Phenanthrene | 4.57 | 1.04 | 48.6 | 1.01 | 0.91 | 7420 ± 110 |
| Pyrene | 4.88 | 0.08 | 21.3 | 0.08 | 0.08 | 6502 ± 740 |

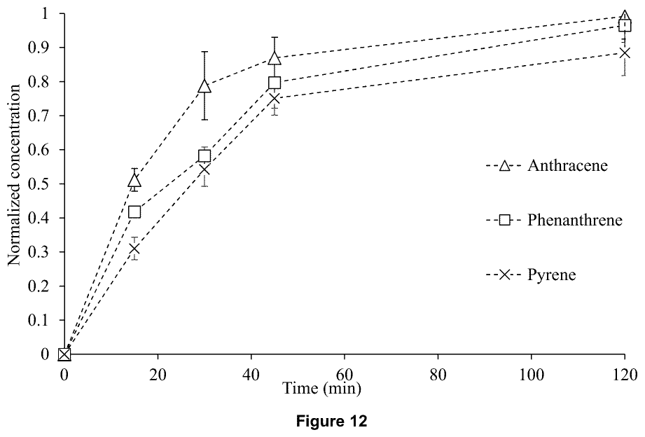

FIG. 12 shows the release of phenanthrene, pyrene from the silicone O-rings into the medium. These results were representative of the PAHs behavior, with the release being sufficiently rapid so that maximum concentrations were reached less than 45 minutes for all components. For naphthalene, after about 4 h, the water concentration decreased due to the volatility of this compound at 15±1° C. (not shown here).

### Characterization of Jellyfish-Like Device Compartment

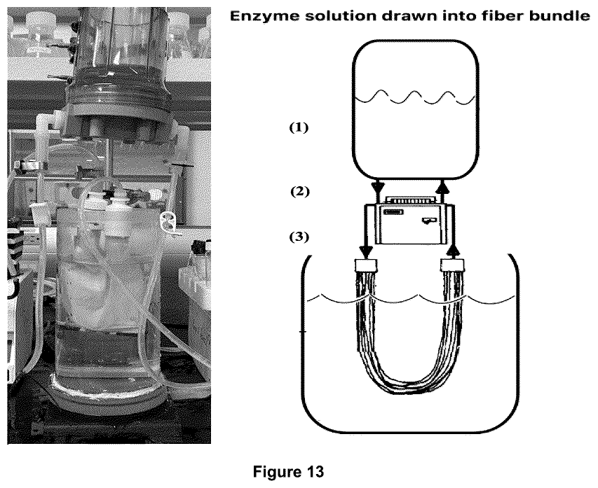

As mentioned previously, jellyfish may have a thing to teach us. FIG. 13 shows a schematic of a hollow-fiber reactor system. The body of jellyfish exhibits radial symmetry and is divided into three parts: the umbrella, the oral arms and the stinging tentacles.⁴⁰ electrospinning has been utilized to synthesize the flexible parts. The main idea behind designing the oral arms is to have flexible membranes with a high surface area. It also serves as membrane adsorbers that facilitate the mass transfer of target substrates from the aqueous phase to membranes. The idea behind designing the tentacles is to have hollow fibers immobilized enzymes that are impermeable to the enzymes but permeable to contaminants resulting in simultaneous adsorption and detoxification of contaminants.

### Hollow Fiber Membrane Characterization

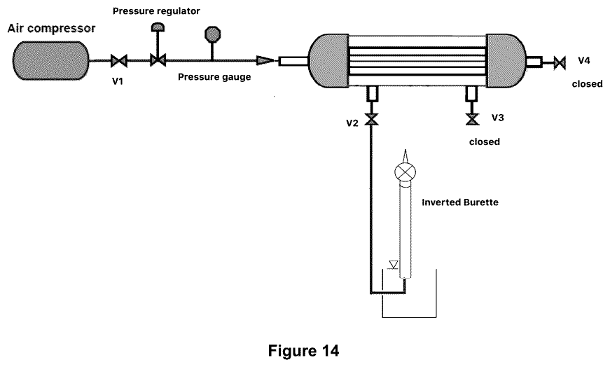

The test to measure diffusion air flow rate was carried out as described elsewhere.⁴¹ Briefly, ultrafiltration (UF) membranes were wetted and pressurized with air to about 30 psi. The shell side is isolated (Valve 2 [V2] is closed and V3 open to measure the volume of displaced air or liquid), and the volume of air flowing through the membrane is measured and compared with values for diffusive airflow for the intact membrane. FIG. 14 shows the setups for the membrane's diffusion flow rate determination. A diffusional flow reading higher than the specification is an indication of a non-integral filter.

### Characterization of the Selected Hollow Fiber Membrane

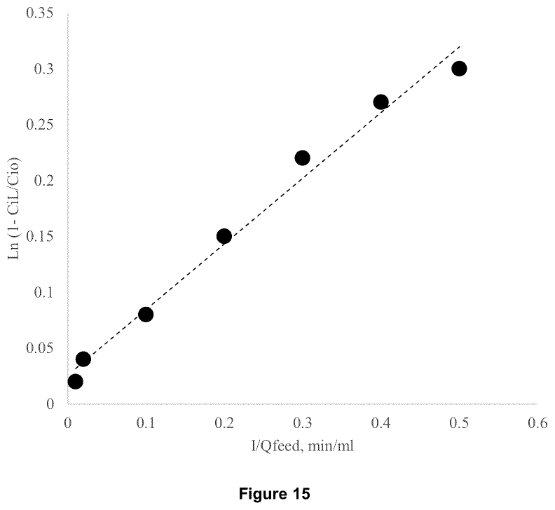

The measured diffusion specification less than 2 SCCM/0.1 m² area showed that the selected module was intact. FIG. 15 shows the plot of PAH model substrate (anthracene) concentration change in the internal recirculated solution vs the reciprocal flow rate to yield the quantity in Eq. (9).

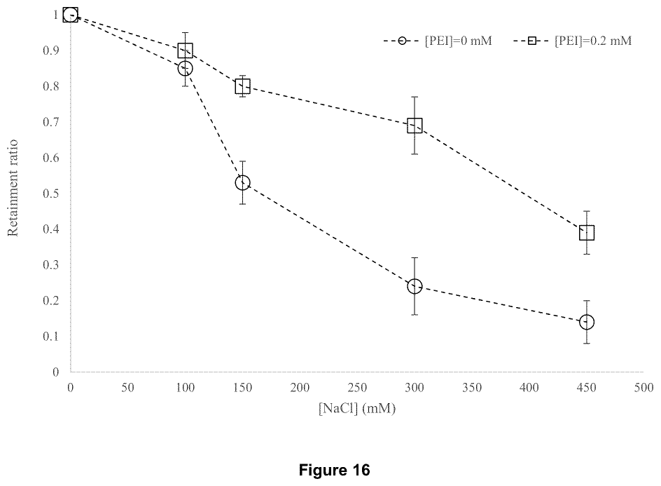

The ratio of permeation coefficient for anthracene used as model substrate (2x 10-3 cm²s⁻¹) to ultrafiltration velocity (54×10⁻³-75×10⁻³ cm ²s⁻¹) obtained in this section will be applied to determine the conversion rate of the substrate at different experimental conditions. The other important permeability characteristic of the selected membrane under experimental condition are coenzyme retention that affects the performance of hollow fiber immobilized enzymes. The membrane reactor system selected benefits a high recycling speed (range of pumping rate from 0.2 ml/min to 200 ml/min) and it was assumed that in this reactor there is no steric problem for interactions between enzymes and coenzymes, and there is a force convection transport through the membrane of the substrate. FIG. 16 shows the retainment ratio of NAD+ retainment ratios in the presence of different concertation of NaCl and PEI.

As shown in FIG. 16, the presence of the high molecular weight polymer polyethyleneimine (PEI) in the reaction medium allowed to enhance retainment ratios for the native coenzyme when NaCl concentration ranged between 100-300 mM. The other consequence of the presence of PEI is the viscosity increase in the enzyme solution and subsequently a decrease of the permeation flux. Our result shows that a 10% decline of flux with respect to the enzyme solution was determined by the presence of a 0.2 mM concentration of PEI.

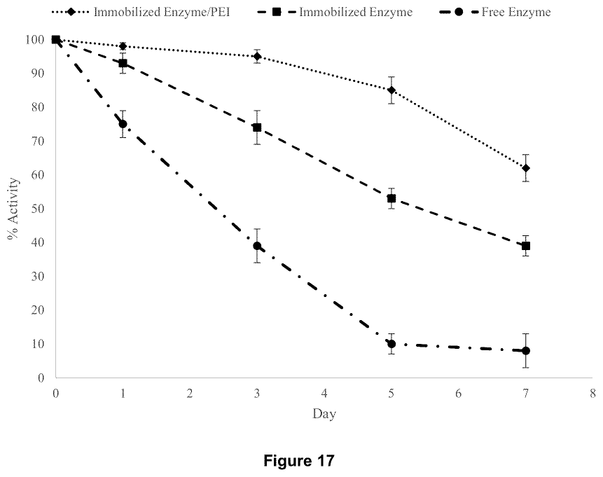

FIG. 17 shows the enzyme activity-time profiles for test solutions containing enzyme cocktail 0.2 mM PEI which were circulated through the lumen of hydrophilic modified polyethersulfone (mPES) hollow fiber beaker containing 500 ml of 20 ppm anthracene in 150 mM saltwater, pH 6.8. Each day saltwater was removed from the beaker and replaced by a fresh substrate solution. A parallel control experiment with native enzyme solution was also run. As shown in FIG. 17, the enzyme solution containing 0.2 mM PEI retained 90% activity for 4 days whereas the free soluble enzyme rapidly deteriorated. Moreover, the stability enhancement resulting from the addition of PEI might be attributed to the entrapment of coenzyme inside the lumen. Thus, it appears feasible to combine in this way the stability advantage of the immobilized enzyme with the semi permeability properties of hollow fibers.³³

### Reactor Performance for Removal of the Model Substrate

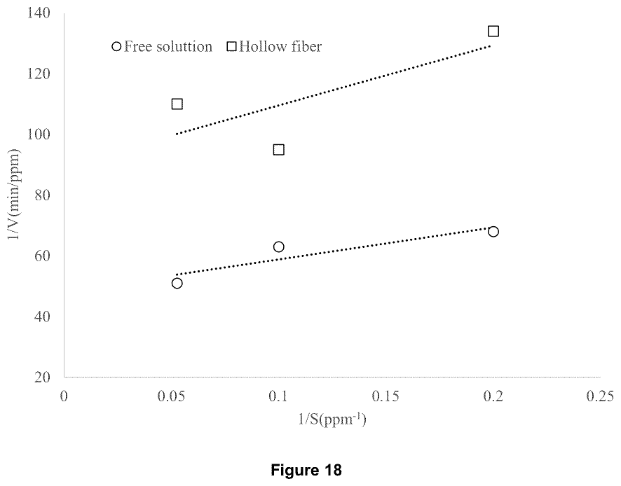

For kinetic analysis of such vertical reactor, enzyme and substrate solution recycle was modeled as a two-compartment membrane reactor separated by an ultrafiltration membrane. The solutions contained in the enzyme compartment (II) and the substrate compartment (I) are assumed to be fully mixed. Reaction rates at various substrate concentrations were measured in free solution and at a recirculating flow rate of 200 ml/min. The Lineweaver-Burk equation was applied to calculate Km of soluble enzyme and hollow fiber immobilized enzyme through the linear fitting. The observed values for the anthracene relate to a multienzyme system of target enzymes. Biotic and abiotic controls are used in the same conditions to distinguish between adsorption and biodegradation for anthracene removal. Two parallel control experiments with inactivated enzyme and without solution circulation were also run. As shown in FIG. 18, the hollow fiber immobilized enzyme (2.24 ppm) was slightly higher than that of the soluble enzyme (2.18 ppm) which might be attributed to the internal diffusion resistance.

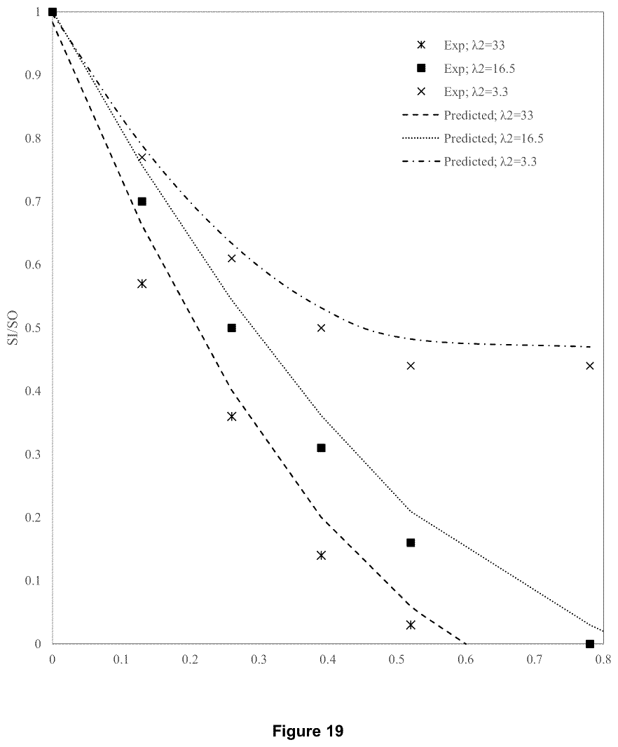

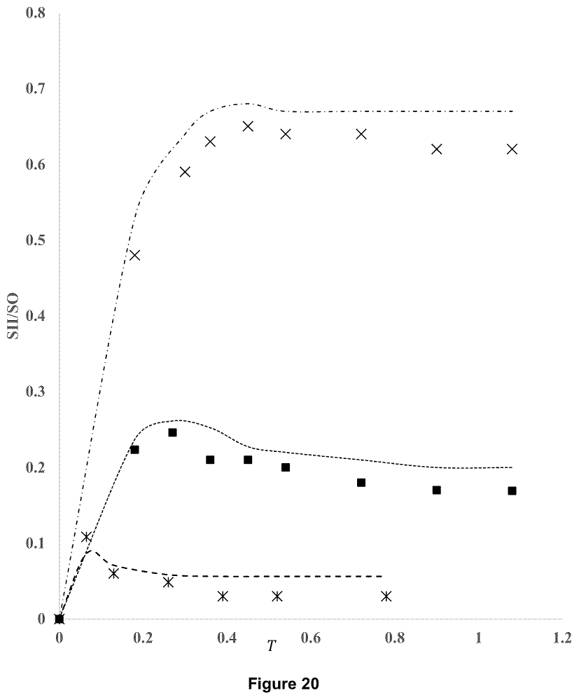

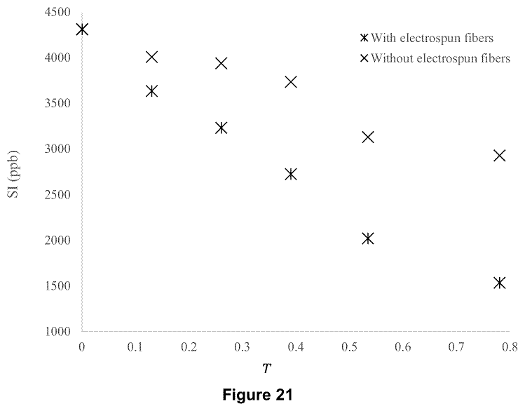

FIGS. 19 and 20 show the simulation results of the anthracene concentration in compartment I and II decreasing with time for two different amounts of enzyme loading in the lumen side (changing A2). Initial substrate concentration is assumed to be 1mM, λ²=3.3, 16.5 and 33 correspond to enzyme concentrations of X, X/5, X/10, respectively level used experimentally. During the batch operation, when the ultrafiltration swing occurs, the parameter is larger than zero and the contribution by diffusion is negligible. As can be seen in FIG. 21, increasing the enzyme concentration increases substrate concentration decay, both experimentally and theoretically. Moreover, substrate concentration in compartment I decays faster in the case of high A which is the measure of reaction rate over diffusion rate. At λ²=33, substrate concentration on the enzyme side nearly equals zero as the enzyme load increases; however, at λ2=3.3 the decay does not follow the exponential decline curve. The good agreement between predicted and measured rates means that the model well describes the recirculated hollow fiber reactor with ultrafiltration swing.

To investigate the effect of electrospun nanofibers on the performance of the reactor system, it has been applied in a recycled hollow fiber enzyme reactor containing oil-water emulsion (e.g., water, anthracene, and lubricant oil) in compartment I (FIG. 21). FIG. 21 shows the removal of the substrate with and without using electrospun nanofibers. The addition of lubricant oil and the behavior of the emulsion filtration with the hydrophilic membranes when the emulsion passes through the membrane might change the apparent ultrafiltration velocity (cm/min) and volume change in each compartment.

A great deal of effort has been made to decrease the tendency of membrane fouling through the application of different techniques to improve membrane properties (e.g., pore size, hydrophilicity, etc.) or pre-treatment of feed stream (e.g., flocculation and microfiltration (MF)). Thus, it is important to take care of the particle loading before the feed gets to membrane units⁴².

### REFERENCES

Irwin, R. J., Mouwerik, M. V., Stevens, L., Seese, M. D., & Basham, W. (1997). Environmental contaminants encyclopedia, naphthalene entry. National Park Service, 1-80.

Minnesota Department of Health. (2019). Anthracene and Groundwater. https://www.health.state.mn.us/communities/environment/risk/docs/guidance/gw/anthrac eneinfo.pdf

1. Hein, F. J., Heavy oil and oil (tar) sands in North America: An overview & summary of contributions. Natural Resources Research 2006, 15, (2), 67-84.

2. Miri, S.; Naghdi, M.; Rouissi, T.; Kaur Brar, S.; Martel, R., Recent biotechnological advances in petroleum hydrocarbons degradation under cold climate conditions: A review. Critical Reviews in Environmental Science and Technology 2019, 49, (7), 553-586.

3. Kadri, T.; Rouissi, T.; Magdouli, S.; Brar, S. K.; Hegde, K.; Khiari, Z.; Daghrir, R.; Lauzon, J.-M., Production and characterization of novel hydrocarbon degrading enzymes from Alcanivorax borkumensis. International Journal of Biological Macromolecules 2018, 112, 230-240.

4. Liu, Z., Benzene and toluene biodegradation with different dissolved oxygen concentrations. Arizona State University: 2015.

5. da Silva, M. L. B.; Alvarez, P. J., Indole-based assay to assess the effect of ethanol on Pseudomonas putida F1 dioxygenase activity. Biodegradation 2010, 21, (3), 425-430.

6. Di Gennaro, P.; Rescalli, E.; Galli, E.; Sello, G.; Bestetti, G., Characterization of Rhodococcus opacus R7, a strain able to degrade naphthalene and o-xylene isolated from a polycyclic aromatic hydrocarbon-contaminated soil. Research in microbiology 2001, 152, (7), 641-651.

7. Miri, S.; Davoodi, S. M.; Brar, S. K.; Rouissi, T.; Sheng, Y.; Martel, R., Psychrozymes as novel tools to biodegrade p-xylene and potential use for contaminated groundwater in the cold climate. Bioresource Technology 2021, 321, 124464.

8. Balashova, N.; Stolz, A.; Knackmuss, H.; Kosheleva, I.; Naumov, A.; Boronin, A., Purification and characterization of a salicylate hydroxylase involved in 1-hydroxy-2-naphthoic acid hydroxylation from the naphthalene and phenanthrene-degrading bacterial strain Pseudomonas putida B5202-P1. Biodegradation 2001, 12, (3), 179-188.

9. Sze, I.; Dagley, S., Properties of salicylate hydroxylase and hydroxyquinol 1, 2-dioxygenase purified from Trichosporon cutaneum. Journal of bacteriology 1984, 159, (1), 353-359.

10. Jardine, J.; Stoychev, S.; Mavumengwana, V.; Ubomba-Jaswa, E., Screening of potential bioremediation enzymes from hot spring bacteria using conventional plate assays and liquid chromatography-Tandem mass spectrometry (Lc-Ms/Ms). Journal of environmental management 2018, 223, 787-796.

11. Ma, H.; Liao, H.; Dellisanti, W.; Sun, Y.; Chan, L. L.; Zhang, L., Characterizing the Host Coral Proteome of Platygyra carnosa Using Suspension Trapping (S-Trap). Journal of Proteome Research 2021.

12. Van Hamme, J. D.; Ward, O. P., Physical and metabolic interactions of Pseudomonas sp. strain JA5-B45 and Rhodococcus sp. strain F9-D79 during growth on crude oil and effect of a chemical surfactant on them. Applied and environmental microbiology 2001, 67, (10), 4874-4879.

13. Wald, J.; Hroudova, M.; Jansa, J.; Vrchotova, B.; Macek, T.; Uhlik, O., Pseudomonads rule degradation of polyaromatic hydrocarbons in aerated sediment. Frontiers in microbiology 2015, 6, 1268.

14. Margesin, R.; Schinner, F.; Marx, J.-C.; Gerday, C., Psychrophiles: from biodiversity to biotechnology. Springer: 2008.

15. Jouanneau, Y.; Meyer, C.; Jakoncic, J.; Stojanoff, V.; Gaillard, J., Characterization of a naphthalene dioxygenase endowed with an exceptionally broad substrate specificity toward polycyclic aromatic hydrocarbons. Biochemistry 2006, 45, (40), 12380-12391.

16. Parales, R. E.; Emig, M. D.; Lynch, N. A.; Gibson, D. T., Substrate specificities of hybrid naphthalene and 2, 4-dinitrotoluene dioxygenase enzyme systems. Journal of Bacteriology 1998, 180, (9), 2337-2344.

17. Mawad, A. M.; Abdel-Mageed, W. S.; Hesham, A. E.-L., Quantification of naphthalene dioxygenase (NahAC) and catechol dioxygenase (C23O) catabolic genes produced by phenanthrene-degrading Pseudomonas fluorescens AH-40. Current Genomics 2020, 21, (2), 111-118.

18. Singleton, D. R.; Hu, J.; Aitken, M. D., Heterologous expression of polycyclic aromatic hydrocarbon ring-hydroxylating dioxygenase genes from a novel pyrene-degrading betaproteobacterium. Applied and environmental microbiology 2012, 78, (10), 3552-3559.

19. Voth, W.; Jakob, U., Stress-activated chaperones: a first line of defense. Trends in biochemical sciences 2017, 42, (11), 899-913.

20. Penning, T. M.; Burczynski, M. E.; Hung, C.-F.; McCoull, K. D.; Palackal, N. T.; Tsuruda, L. S., Dihydrodiol dehydrogenases and polycyclic aromatic hydrocarbon activation: generation of reactive and redox active o-quinones. Chemical research in toxicology 1999, 12, (1), 1-18.

21. Tomas-Gallardo, L.; Canosa, I.; Santero, E.; Camafeita, E.; Calvo, E.; López, J. A.; Floriano, B., Proteomic and transcriptional characterization of aromatic degradation pathways in Rhodoccocus sp. strain TFB. Proteomics 2006, 6, (S1), S119-S132.

22. Borisova, R. B. Isolation of a Rhodococcus soil bacterium that produces a strong antibacterial compound. East Tennessee State University, 2011.

23. Miri, S.; Perez, J. A. E.; Brar, S. K.; Rouissi, T.; Martel, R., Sustainable production and co-immobilization of cold-active enzymes from Pseudomonas sp. for BTEX biodegradation. Environmental Pollution 2021, 285, 117678.

24. Altschul, S. F.; Gish, W.; Miller, W.; Myers, E. W.; Lipman, D. J., Basic local alignment search tool. Journal of molecular biology 1990, 215, (3), 403-410.

25. Tamura, K.; Peterson, D.; Peterson, N.; Stecher, G.; Nei, M.; Kumar, S., MEGA: molecular evolutionary genetics analysis using maximum likelihood, evolutionary distance, and maximum parsimony methods. Molecular biology and evolution 2011, 28, (10), 2731-2739.

26. ASTM, D., Standard test method for particle-size analysis of soils. 2007.

27. Martel, R.; Gelinas, P. J., Surfactant solutions developed for NAPL recovery in contaminated aquifers. Ground Water 1996, 34, (1), 143-155.

28. Singh, P.; Kanwar, R. S., Preferential solute transport through macropores in large undisturbed saturated soil columns. Journal of Environmental Quality 1991, 20, (1), 295-300.

29. Davoodi, S. M.; Brar, S. K.; Galvez-Cloutier, R.; Martel, R., Performance of packed and fluidized bed columns for the removal of unconventional oil using modified dolomite. Fuel 2021, 285, 119191.

30. Beaulieu, M.; contaminés, Q. S. d. I., Guide d'intervention: protection des sols et réhabilitation des terrains contaminés. Ministère du Développement durable, de l'Environnement et de la Lutte contre . . . : 2016.

31. Yu, Q.; Zhang, Y.; Wang, H.; Brash, J.; Chen, H., Anti-fouling bioactive surfaces. Acta biomaterialia 2011, 7, (4), 1550-1557.

32. Obón, J.; Almagro, M. J.; Manjón, A.; Iborra, J., Continuous retention of native NADP (H) in an enzyme membrane reactor for gluconate and glutamate production. Journal of biotechnology 1996, 50, (1), 27-36.

33. Chambers, R.; Cohen, W.; Baricos, W., [23] Physical immobilization of enzymes by hollow-fiber membranes. In Methods in enzymology, Elsevier: 1976; Vol. 44, pp 291-317.

34. Kleinhans, F., Membrane permeability modeling: Kedem-Katchalsky vs a two-parameter formalism. Cryobiology 1998, 37, (4), 271-289.

35. Fournier, R. L., Basic transport phenomena in biomedical engineering. CRC press: 2017.

36. Li, Z.; Cabana, H.; Lecka, J.; Brar, S. K.; Galvez, R.; Bellenger, J.-P., Efficiencies of selected biotreatments for the remediation of PAH in diluted bitumen contaminated soil microcosms. Biodegradation 2021, 1-14.

37. Butler, J. D.; Parkerton, T. F.; Letinski, D. J.; Bragin, G. E.; Lampi, M. A.; Cooper, K. R., A novel passive dosing system for determining the toxicity of phenanthrene to early life stages of zebrafish. Science of the Total Environment 2013, 463, 952-958.

38. Smith, K. E.; Oostingh, G. J.; Mayer, P., Passive dosing for producing defined and constant exposure of hydrophobic organic compounds during in vitro toxicity tests. Chemical research in toxicology 2010, 23, (1), 55-65.

39. Gilbert, D.; Mayer, P.; Pedersen, M.; Vinggaard, A. M., Endocrine activity of persistent organic pollutants accumulated in human silicone implants—Dosing in vitro assays by partitioning from silicone. Environment international 2015, 84, 107-114.

40. Yu, J.; Li, X.; Pang, L.; Wu, Z., Design and attitude control of a novel robotic jellyfish capable of 3D motion. Sci. China Inf. Sci. 2019, 62, (9), 194201:1-194201:3.

41. Farahbakhsh, K.; Smith, D., Estimating air diffusion contribution to pressure decay during membrane integrity tests. Journal of membrane science 2004, 237, (1-2), 203-212.

42. Zuo, G.; Wang, R., Novel membrane surface modification to enhance anti-oil fouling property for membrane distillation application. Journal of membrane science 2013, 447, 26-35.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

What is claimed is:

1. A polyaromatic hydrocarbon (PAH)-degrading enzyme mixture obtained from a culture of PAH-utilizing microorganisms having been grown in presence of one or more enzyme inducers.

2. The PAH-degrading enzyme mixture of claim 1, wherein the culture is a mixed-culture of two or more PAH-utilizing microorganisms.

3. The PAH-degrading enzyme mixture of claim 1, wherein the PAH-utilizing microorganisms are bacteria that are capable of growing with a PAH-containing composition being their only source of carbon.

4. The PAH-degrading enzyme mixture of claim 1, wherein the PAH-utilizing microorganisms are indigenous to a PAH-contaminated soil sample.

5. The PAH-degrading enzyme mixture of claim 1, wherein the PAH-utilizing microorganisms comprise Pseudomonas sp. and/or Rhodococcus sp.

6. The PAH-degrading enzyme mixture of claim 5, wherein the PAH-utilizing microorganisms comprise Pseudomonas sp. URS-5, Pseudomonas sp. URS-6, Pseudomonas sp. URS-8, and/or Rhodococcus sp. URS-10.

7. The PAH-degrading enzyme mixture of claim 1, wherein the one or more enzyme inducers comprise naphthalene, anthracene, phenanthrene, pyrene, benzo[a]pyrene, Dilbit, and/or crude oil.

8. The PAH-degrading enzyme mixture of claim 1, comprising an optimum active enzyme temperature of less than about 30° C., such as less than about 25° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., and any range therein between, such as from about 10° C. to about 15° C.

9. The PAH-degrading enzyme mixture of claim 1, comprising an optimum pH of from about 4 to about 9, such as from about 4, about 5, about 6, about 7, or about 8 to about 5, about 6, about 7, about 8, or about 9, such as about 4, about 5, about 6, about 7, about 8, or about 9, such as about 7.

10. The PAH-degrading enzyme mixture of claim 1, comprising less than about 10% salt or for use in an environment containing less than about 10% salt, such as less than about 9%, less than about 8%, less than about 7%, less than about 6%, less than about 5%, less than about 4%, less than about 3%, less than about 2%, or less than about 1% salt.

11. The PAH-degrading enzyme mixture of claim 1, wherein the enzymes are adsorbed on soil particles.

12. The PAH-degrading enzyme mixture of claim 1, further comprising a dispersing aid, such as a surfactant or biosurfactant, such as a rhamnolipid.

13. The PAH-degrading enzyme mixture of claim 1, wherein the enzymes comprise naphthalene dioxygenase, naphthalene cis-dihydridiol dehydrogenase, dihydrodiol dehydrogenase, catechol 1,2 dioxygenase, catechol 2,3 dioxygenase, salicylaldehyde dehydrogenase, 1-hydroxy-2-naphthoate hydroxylase, salicylate hydroxylase, trans-2-carboxybenzalpyruvate hydratase-aldolase, lipase, toluene monooxygenase, lignin peroxidase, manganese peroxidase, esterase, and/or laccase.

14. The PAH-degrading enzyme mixture of claim 1, further comprising one or more species of live PAH-utilizing organisms.

15. A method for tailoring a polyaromatic hydrocarbon (PAH)-degrading enzyme mixture for a selected contaminated site for bioremediation, the method comprising:
isolating indigenous PAH-utilizing microorganisms from the contaminated site,
culturing the microorganisms in the presence of one or more enzyme inducers,
extracting enzymes from the culture, and
applying the enzymes to the contaminated site.

16. The method of claim 15, wherein the contaminated site comprises soil.

17. The method of claim 15, wherein the contaminated site comprises less than about 10% salt, such as less than about 9%, less than about 8%, less than about 7%, less than about 6%, less than about 5%, less than about 4%, less than about 3%, less than about 2%, or less than about 1% salt.

18. The method of claim 15, further comprising applying one or more species of live PAH-utilizing organisms to the contaminated site.

19. A jellyfish-like device for bioremediation of a contaminated water site, the device comprising:
an enzyme reservoir containing PAH-degrading enzymes, and
a hollow fiber module in fluid communication with the enzyme reservoir,
wherein the hollow fiber module is permeable to PAHs but not to the PAH-degrading enzymes.

20. The device of claim 19, wherein the hollow fiber module comprises hydrophilic modified polyethersulfone (mPES) hollow fibers.

21. The device of claim 19, wherein the hollow fiber module comprises from about 1 to about 100 fibers, wherein each fiber independently has an internal diameter of from about 0.1 mm to about 10 mm, wherein each fiber independently has a length of from about 10 to about 100 cm, wherein each fiber independently has a wall thickness of from about 0.01 mm to about 1 mm, and/or wherein each fiber independently has a total surface area of from about 10 cm2 to about 100 cm².

22. The device of claim 19, further comprising a recycling pump, wherein the recycling pump optionally operates at a speed of from about 1 ml/min to about 100 ml/min, such as from about 1, about 2, about 3, about 4, about 5, about 6, about 7, about 8, about 9, about 10, about 20, about 30, about 40, about 50, about 60, about 70, about 80, or about 90 to about 2, about 3, about 4, about 5, about 6, about 7, about 8, about 9, about 10, about 20, about 30, about 40, about 50, about 60, about 70, about 80, about 90, or about 100, ml/min, such as about 50 ml/min.

23. A mobile production plant for producing the PAH-degrading enzymes of claim 1, the production plant comprising a fermenter for culturing microorganisms, a bioreactor for enzyme production, and an ultrasonic device for producing cell extracts.

24. The mobile production plant of claim 23, wherein the fermenter cultures the microorganisms at room temperature.

25. The mobile production plant of claim 23, further comprising a power generator.

26. The mobile production plant of claim 23, further comprising an oxygen source such as an oxygen cylinder.

27. The mobile production plant of claim 23, wherein the mobile production plant is provided on a trailer.
